(19) United States
(12) Patent Application Publication
(10) Pub. No.: US 2010/0104625 A1
(43) Pub. Date: Apr. 29, 2010

(54) BIODEGRADABLE COMPOSITIONS AND MATERIALS

(75) Inventors: David Aaron Putnam, Ithaca, NY (US); Sara Yazdi, Needham, MA (US)

Correspondence Address: CHOATE, HALL & STEWART LLP, TWO INTERNATIONAL PLACE, BOSTON, MA, 02110, US

(73) Assignee: CORNELL UNIVERSITY, Ithaca, NY (US)

(21) Appl. No.: 12/527,222

(22) PCT Filed: Feb. 15, 2008
(86) PCT No.: PCT/US2008/054106

§ 371 Date: Dec. 10, 2009

Related U.S. Application Data
(60) Provisional application No. 60/901,964, filed on Feb. 16, 2007.

(51) Int. Cl. A61K 9/127; A61K 9/14; A61K 38/02; A61K 31/7088; A61K 35/00; A61K 38/16; A61K 38/43; A61K 31/573

(52) U.S. Cl. 424/450; 424/484; 514/2; 514/44 R; 424/93.1; 514/12; 424/94.1; 514/180; 977/906

(57) ABSTRACT

The present invention provides compounds of Formula I, materials comprising one or more compounds of Formula I and one or more biologically active agents, pharmaceutical compositions comprising an inventive material and a pharmaceutically acceptable excipient, methods of making compounds of Formula I, methods of making inventive materials, and methods of using pharmaceutical compositions comprising an inventive material and a pharmaceutically acceptable excipient.

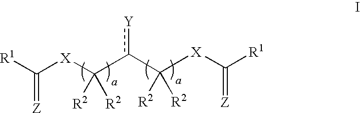

### BACKGROUND OF THE INVENTION

A diverse collection of natural and synthetic biodegradable materials has been studied as controlled drug delivery systems. For example, natural biodegradable polymers, such as human serum albumin and collagen, as well as synthetic biodegradable polymers, such as the polyesters based on lactic and glycolic acids, have been used for a range of applications (Duncan, Nat. Rev. Drug Discov. (2003) 2:347-360; Hunter and Moghimi, Drug Discov. Today (2002) 7:998-1001; Langer, Science (2001) 293:58-59; Muller and Keck, J. Biotechnol. (2004) 113:151-170; Rosen and Abribat, Nat. Rev. Drug Discov. (2005) 4:381-385).

Solid lipids are a class of materials that also have promise in controlled drug delivery systems. Since their origination in the 1990's, solid lipid particles have received considerable attention as new drug carrier systems. Solid lipid particles are attractive in that they can be derived from physiological lipids and have well defined molecular weights. Additionally, their building blocks can be chosen from a diverse population of structures, such as, for example, glycerols, fatty acids, and triglycerides. Solid lipid microparticles (SLM) have been used to encapsulate various drugs such as clobetasol, GnRH antagonist, and hepatitis B surface antigen (Hu et al., Int. J. Pharm. (2002) 239:121-128; DelCurto et al., J. Control. Release (2003) 89:297-310; Pandey and Khuller, Tuberculosis (2005) 85:227-234; Saraf et al., Vaccine (2006) 24:45-56; Gavini et al., Pharm. Dev. Technol. (2005) 10:479-487). Despite the ongoing growth of SLMs as drug delivery carriers, diglycerides remain an untapped resource in comparison to other members of the lipid family.

### SUMMARY OF THE INVENTION

The present invention provides compounds of Formula I and pharmaceutically acceptable salts, prodrugs or derivatives thereof; and materials (e.g., for example, particles, films, coatings, micelles, and the like) and pharmaceutical compositions comprising them. For example, the present invention provides materials comprising one or more compounds of Formula I, or a pharmaceutically acceptable salt, prodrug or derivative thereof; materials comprising one or more compounds of Formula I and one or more biologically active agents; and materials comprising one or more compounds of Formula I and one or more diagnostic agents. The present invention also provides pharmaceutical compositions comprising a compounds of Formula I and a pharmaceutically acceptable excipient; and pharmaceutical compositions comprising an inventive material and a pharmaceutically acceptable excipient. The present invention also provides methods of making compounds of Formula I and methods of making materials comprising them. Additionally, the present invention provides methods of using pharmaceutical compositions comprising an inventive material and a pharmaceutically acceptable excipient. In certain embodiments, the inventive materials are solid lipid microparticles (SLM) for drug delivery. In certain embodiments, compounds of Formula I and/or the inventive materials are biodegradable. In certain embodiments, compounds of Formula I and/or the inventive materials are biocompatible.

For example, in one aspect, the present invention provides a compound of Formula I, or a pharmaceutically acceptable salt, prodrug, or derivative thereof:

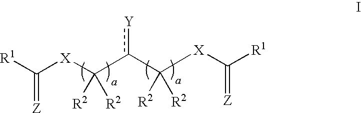

wherein:

[symbol] Y is (═O), —OR^{O}, (═S), —SR^{S}, (═NR^{N}), or —N(R^{N})₂, wherein

R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group;

R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thio protecting group; and

each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

each instance of X is, independently, O, S, or N(R^{N});

each instance of Z is, independently, O, S, or N(R^{N});

each instance of R¹ is, independently, cyclic or acyclic, substituted or unsubstituted aliphatic; cyclic or acyclic, substituted or unsubstituted heteroaliphatic; substituted or unsubstituted aryl; or substituted or unsubstituted heteroaryl;

each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and

each instance of a is, independently, an integer between 1 to 6, inclusive.

In certain embodiments, each instance of R¹ is, independently, cyclic or acyclic, substituted or unsubstituted C₁₋₃₂ aliphatic. In certain embodiments, each instance of R¹ is, independently, cyclic or acyclic, substituted or unsubstituted C₁₋₃₂ heteroaliphatic. In certain embodiments, each instance of R¹ is independently, cyclic or acyclic, substituted or unsubstituted C₁₋₃₂ alkynyl. In certain embodiments, each instance of R¹ is independently, cyclic or acyclic, substituted or unsubstituted C₁₋₃₂ alkenyl. In certain embodiments, each instance of R¹ is, independently, cyclic or acyclic, substituted or unsubstituted C₁₋₃₂ alkyl.

In certain embodiments, R¹ is a C₂₋₃₂-fatty acid substituent. Exemplary fatty acids include, but are not limited to, saturated fatty acids, monoenoic fatty acids, polyunsaturated fatty acids, methyl-branched fatty acids, ring-containing fatty acids, methoxy fatty acids, thia fatty acids, keto fatty acids, and oxo fatty acids.

In another aspect, the present invention provides a material (e.g., for example, a particle, film, coating, micelle, and the like) comprising one or more compounds of Formula I and one or more biologically active agents.

In another aspect, the present invention provides a pharmaceutical composition for delivery of a biologically active agent, said composition comprising one or more compounds of Formula I and a pharmaceutically acceptable excipient.

In yet another aspect, the present invention provides a pharmaceutical composition for delivery of a biologically active agent, said composition comprising an inventive material and a pharmaceutically acceptable excipient.

In certain embodiments, a biologically active agent is a therapeutic cell, a small organic molecule (e.g., hydrophobic and/or hydrophilic drug compounds), an amino acid, a dipeptide, a polypeptide, a protein, an enzyme, a carbohydrate, a monosaccharide, a disaccharide, an oligosaccharide, a polysaccharide, a nucleoprotein, a mucoprotein, a lipoprotein, a small molecule linked to a protein, a glycoproteins, a steroid, a nucleic acid, DNA, RNA, a nucleotide, a nucleoside, an oligonucleotide, an antisense oligonucleotide, a lipid, a hormone, a vitamin, a metal, a transition metal, an organometal, or a combination thereof.

In certain aspects, the inventive material is biodegradable. In certain aspects, the inventive material is biocompatible. In certain embodiments, the inventive material, upon biodegrading, slowly releases the biologically active agent.

In another aspect, the present invention provides a method of making a compound of Formula I, the method comprising the steps of:

(i) providing a compound of Formula II:

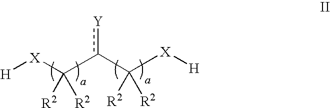

wherein:

[symbol] Y is (═O), —OR^{O}, (═S), —SR^{S}, (═NR^{N}), or —N(R^{N})₂, wherein

R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group;

R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thio protecting group; and

each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

each instance of X is, independently, O, S, or N(R^{N});

each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and

each instance of a is, independently, an integer between 1 to 6, inclusive;

(ii) providing one or more compounds of Formula III:

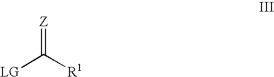

wherein:

Z is, independently, O, S, or N(R^{N}), wherein R^{N} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

R¹ is cyclic or acyclic, substituted or unsubstituted aliphatic; cyclic or acyclic, substituted or unsubstituted heteroaliphatic; substituted or unsubstituted aryl; or substituted or unsubstituted heteroaryl; and

LG is a suitable leaving group;

(iii) mixing a compound of Formula II with one or more compounds of Formula III to provide a compound of Formula I.

In other aspects, the present invention provides a method of making an inventive material (e.g., a particle, a film, a coating, a micelle), the method comprising the steps of:

(i) providing one or more compounds of the Formula I;

(ii) providing one or more biologically active agents;

(iii) adding one or more compounds of Formula I and one or more biologically active agents to a suitable solution to provide a mixture; and

(iv) stirring the mixture of the compound and one or more biologically active agents with evaporation of the suitable solution.

In certain embodiments, the biologically active agent is non-covalently associated with one or more compounds of Formula I. In certain embodiments, the biologically active agent is non-covalently encapsulated by one or more compounds of Formula I.

In another aspect, the present invention provides a method of using a pharmaceutical composition comprising the inventive material and a pharmaceutically acceptable excipient, the method comprising administering to a subject in need thereof a therapeutically effective amount of the pharmaceutical composition.

These and other aspects of the present invention will be apparent from the following description of certain embodiments, from the examples, and from the claims.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that a compound, as described herein, may be substituted with any number of substituents or functional moieties. In general, the term “substituted” whether preceded by the term “optionally” or not, and substituents contained in Formulas of this invention, refer to the replacement of hydrogen radicals in a given structure with the radical of a specified substituent. As appreciated by one skilled in this art, one can change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

In certain embodiments, substituents include, but are not limited to, any of the substituents described herein, that result in the formation of a stable moiety. Exemplary substituents include aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds.

The term “stable”, as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a group having the general Formula —C(═O)R, where R is hydrogen, halogen, hydroxy, thio, amino, optionally substituted aliphatic, optionally substituted heteroaliphatic, optionally substituted alkyl, optionally substituted alkenyl, optionally substituted alkynyl, optionally substituted aryl, alkyloxy, alkylthioxy, alkylamino, dialkylamino, arylamino, diarylamino, optionally substituted aryl, optionally substituted heteroaryl, or optionally substituted heterocycyl. Exemplary acyl groups include aldehydes, carboxylic acids, ketones (such as an acetyl group [—(C═O)CH₃], esters, amides, carbonates, carbamates, and ureas. Substituents include, but are not limited to, any of the substituents described herein, that result in the formation of a stable moiety. In certain embodiments, the aliphatic, heteroaliphatic, alkyl, alkenyl, alkynyl, aryl, alkyloxy, alkylthioxy, amino, heteroaryl, or heterocycyl moieties present on the acyl group are further substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. Substituents include, but are not limited to, any of the substituents described herein, that result in the formation of a stable moiety. In certain embodiments, aliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “alkyl” as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-32 carbon atoms. In some embodiments, the alkyl group employed in the invention contains 1-26 carbon atoms. In another embodiment, the alkyl group employed contains 1-18 carbon atoms. In another embodiment, the alkyl group employed contains 2-18 carbon atoms. In another embodiment, the alkyl group employed contains 4-18 carbon atoms. In another embodiment, the alkyl group employed contains 6-18 carbon atoms. In another embodiment, the alkyl group employed contains 8-18 carbon atoms. In still other embodiments, the alkyl group contains 10-18 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl (C₁₂), tridecyl (C₁₃), tetradecyl (C₁₄), pentadecyl (C₁₅), hexadecyl (C₁₆), heptadecyl (C₁₇), octadecyl (C₁₈), nonadecyl (C₁₉), eicosyl (C₂₀), uncosyl (C₂₁), docosyl (C₂₂), tricosyl (C₂₃), tetracosyl (C₂₄), pentacosyl (C₂₅), hexacosyl (C₂₆), and the like, which may bear one or more substitutents. Substituents include, but are not limited to, any of the substituents described herein, that result in the formation of a stable moiety. In certain embodiments, alkyl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “hydroxy,” or “hydroxyl,” as used herein, refers to a group of the Formula (—OH). An “optionally substituted hydroxy” refers to a group of the Formula (—OR), wherein R can be hydrogen, or any substitutent. Substituents include, but are not limited to, any of the substituents described herein, that result in the formation of a stable moiety. In certain embodiments, the hydroxyl group is substituted by independent replacement of the hydrogen atom thereon with a different moiety, including, but not limited to aliphatic; heteroaliphatic; aryl; heteroaryl; arylalkyl; heteroarylalkyl; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —S(O)₂Rₓ; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

A “suitable hydroxyl protecting group,” or a “protected hydroxyl group,” as used herein, is well known in the art and include those described in detail in Protecting Groups in Organic Synthesis, T. W. Greene and P. G. M. Wuts, 3rd edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The term “alkoxy” as used herein refers to an alkyl group, as defined herein, attached to the parent molecular moiety through an oxygen atom (i.e., alkyl-O—). Examples of alkoxy groups include, but are not limited to, methoxy, ethoxy, propoxy, isopropoxy, n-butoxy, tert-butoxy, i-butoxy, sec-butoxy, neopentoxy, n-hexoxy, n-heptoxy, n-octyloxy, n-decyloxy, n-undecyloxy, dodecyloxy (C₁₂), tridecyloxy (C₁₃), tetradecyloxy (C₁₄), pentadecyloxy (C₁₅), hexadecyloxy (C₁₆), heptadecyloxy (C₁₇), octadecyloxy (C₁₈), nonadecyloxy (C₁₉), eicosyloxy (C₂₀), heneicosyloxy (C₂₁), docosyloxy (C₂₂), tricosyloxy (C₂₃), tetracosyloxy (C₂₄), pentacosyloxy (C₂₅), hexacosyloxy (C₂₆), and the like. Substituents include, but are not limited to, any of the substituents described herein, that result in the formation of a stable moiety. In certain embodiments, alkyl moiety of the alkyloxy group is further substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “alkenyl” denotes a monovalent group derived from a hydrocarbon moiety having at least one carbon-carbon double bond by the removal of a single hydrogen atom. In certain embodiments, the alkenyl group employed in the invention contains 2-32 carbon atoms. In certain embodiments, the alkenyl group employed in the invention contains 2-26 carbon atoms. In some embodiments, the alkenyl group employed in the invention contains 2-18 carbon atoms. In another embodiment, the alkenyl group employed contains 4-18 carbon atoms. In another embodiment, the alkenyl group employed contains 6-18 carbon atoms. In another embodiment, the alkenyl group employed contains 8-18 carbon atoms. In another embodiment, the alkenyl group employed contains 10-18 carbon atoms. Alkenyl groups include, for example, ethenyl, propenyl, butenyl, 1-methyl-2-buten-1-yl, and the like. Substituents include, but are not limited to, any of the substituents described herein, that result in the formation of a stable moiety. In certain embodiments, alkenyl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “alkynyl” as used herein refers to a monovalent group derived form a hydrocarbon having at least one carbon-carbon triple bond by the removal of a single hydrogen atom. In certain embodiments, the alkynyl group employed in the invention contains 2-32 carbon atoms. In certain embodiments, the alkynyl group employed in the invention contains 2-26 carbon atoms. In some embodiments, the alkynyl group employed in the invention contains 2-18 carbon atoms. In another embodiment, the alkynyl group employed contains 4-18 carbon atoms. In another embodiment, the alkynyl group employed contains 6-18 carbon atoms. In another embodiment, the alkynyl group employed contains 8-18 carbon atoms. In another embodiment, the alkynyl group employed contains 10-18 carbon atoms. Representative alkynyl groups include, but are not limited to, ethynyl, 2-propynyl (propargyl), 1-propynyl, and the like. Substituents include, but are not limited to, any of the substituents described herein, that result in the formation of a stable moiety. In certain embodiments, alkynyl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “optionally substituted amino,” as used herein refers to —NH₂, or an —NH₂ group substituted with one, two, or three substituents, and which results in a stable moiety. Substituents include, but are not limited to, any of the substituents described herein, that result in the formation of a stable moiety. In certain embodiments, the amino moiety is substituted by independent replacement of one or more of the hydrogen atoms thereon with one or more moieties including, but not limited to a suitable amino protecting group; aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —OH; —C(O)R_{X}; —CO₂(Rₓ); —CON(Rₓ)₂; —N(Rₓ)₂; —S(O)₂Rₓ; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

A “suitable amino protecting group,” or a “protected amino group,” as used herein, is well known in the art and include those described in detail in Protecting Groups in Organic Synthesis, T. W. Greene and P. G. M. Wuts, 3rd edition, John Wiley & Sons, 1999, the entirety of which is incorporated herein by reference. Suitable amino protecting groups, include, but are not limited to, aralkyl groups, allyl groups, acyl groups, acyloxy groups, and the like. Examples of suitable amino protecting groups include t-butyloxycarbonyl (BOC; —C═O)OtBu), ethyloxycarbonyl (—C(═O)OEt), methyloxycarbonyl (—C(═O)OMe), trichloroethyloxycarbonyl (—C(═O)OCH₂CCl₃), allyloxycarbonyl (-Alloc; —(C═O)OCH₂CH═CH₂), benzyloxycarbonyl (-Cbz; —(C═O)OBn), allyl (—CH₂CH═CH₂), benzyl (-Bn), fluorenylmethylcarbonyl (-Fmoc), formamido, acetyl, chloroacetyl, dichloroacetyl, trichloroacetyl, phenylacetyl, trifluoroacetyl, benzamido, t-butyldiphenylsilyl (TBDMS), toluenesulfonyl (-Ts), and the like. Suitable amino protecting groups also include amines that are substituted with two substituents independently selected from those described above, and further includes cyclic imides, such as phthalimide, maleimide, succinimide, and the like. Suitable di-protected amines also include pyrroles and the like.

The terms “alkylamino, dialkylamino, and trialkylamino” as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. In certain embodiments, the alkyl group contain 1-26 aliphatic carbon atoms. In certain other embodiments, the alkyl group contains 1-18 aliphatic carbon atoms. In yet other embodiments, the alkyl group contains 8-18 aliphatic carbon atoms. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino. Substituents include, but are not limited to, any of the substituents described herein, that result in the formation of a stable moiety. In certain embodiments, the alkyl substituents on the amino moiety are further substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “thio,” or “thiol,” as used herein, refers to a group of the Formula (—SH). An “optionally substituted thiol” refers to a group of the Formula (—SR), wherein R can be hydrogen, or any substitutent. Substituents include, but are not limited to, any of the substituents described herein, that result in the formation of a stable moiety. In certain embodiments, the thiol moiety is substituted by independent replacement of the hydrogen atom present thereon with a different moiety, including, but not limited to a suitable thio protecting group; aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

A “suitable thio protecting group,” or a “protected thiol group,” as used herein, is well known in the art and include those described in detail in Protecting Groups in Organic Synthesis, T. W. Greene and P. G. M. Wuts, 3rd edition, John Wiley & Sons, 1999, the entirety of which is incorporated herein by reference. Examples of suitably protected thiol groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The terms “alkylthioether” and “thioalkoxyl” refer to a saturated (i.e., alkyl-S—) or unsaturated (i.e., alkenyl-S— and alkynyl-S—) group attached to the parent molecular moiety through a sulfur atom. In certain embodiments, the alkyl group contains 1-32 aliphatic carbon atoms. In certain embodiments, the alkyl group contains 1-26 aliphatic carbon atoms. In certain other embodiments, the alkyl group contains 1-18 aliphatic carbon atoms. In yet other embodiments, the alkyl, alkenyl, and alkynyl groups contain 8-18 aliphatic carbon atoms. Examples of thioalkoxyl moieties include, but are not limited to, methylthio, ethylthio, propylthio, isopropylthio, n-butylthio, and the like. Substituents include, but are not limited to, any of the substituents described herein, that result in the formation of a stable moiety. In certain embodiments, thioalkoxyl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aryl,” as used herein, refer to stable aromatic mono- or polycyclic ring system having 3-20 ring atoms, of which all ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Substituents include, but are not limited to, any of the substituents described herein, that result in the formation of a stable moiety. In certain embodiments, aryl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Substituents include, but are not limited to, any of the substituents described herein, that result in the formation of a stable moiety. In certain embodiments, heteroaryl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The terms “halo” and “halogen” as used herein refer to an atom selected from fluorine, chlorine, bromine, and iodine.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “heterocyclic,” or “heterocyclyl,” as used herein, refers to an non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety. In certain embodiments, heterocyclyl moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; ═O, ═S, ═NRₓ, —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

As used herein, the term “pharmaceutically acceptable salt” refers to those salts which are, within the scope of sound medical judgment, suitable for use in contact with the tissues of humans, animals, or plants, without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “prodrug” refers to a derivative of a parent compound that requires transformation within the body in order to release the parent compound. In certain cases, a prodrug has improved physical and/or delivery properties over the parent compound. Prodrugs are typically designed to enhance pharmaceutically and/or pharmacokinetically based properties associated with the parent compound. The advantage of a prodrug can lie in its physical properties, such as enhanced water solubility for parenteral administration at physiological pH compared to the parent compound, or it enhances absorption from the digestive tract, or it may enhance drug stability for long-term storage. In recent years several types of bioreversible derivatives have been exploited for utilization in designing prodrugs. Using esters as a prodrug type for compounds containing a carboxyl or hydroxyl functionality is known in the art as described, for example, in “The Organic Chemistry of Drug Design and Drug Interaction” Richard Silverman, published by Academic Press (1992).

The following definitions are more general terms used throughout the present application:

“Subject”: The term “subject,” as used herein, refers to any animal (e.g., vertebrate, invertebrate) or plant (e.g., crops or agricultural plants such as, for example, corn, wheat and rice, trees, flowers, herbs, bushes, grasses, vines, ferns, and mosses). In certain embodiments, the subject is a mammal. In certain embodiments, the term “subject”, as used herein, refers to a human (e.g., a man, a woman, child, juvenile, adult, and/or senior adult).

The terms “administer,” “administering,” or “administration,” as used herein refers to spraying or coating, implanting, absorbing, ingesting, injecting, or inhaling, the inventive material or compound.

The terms “treat” or “treating,” as used herein, refers to partially or completely alleviating, inhibiting, ameliorating, and/or relieving the disease or condition from which the subject is suffering.

The terms “effective amount” and “therapeutically effective amount,” as used herein, refer to the amount or concentration of a biologically active agent present in a pharmaceutical composition or inventive material of the presently claimed invention, that, when administered to a subject, is effective to at least partially treat a condition from which the subject is suffering.

As used herein, when two entities are “conjugated” to one another they are linked by an indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction is when two entities are covalently connected through a linker group.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effects on the cells (i.e., fewer than about 20% of the cells are killed when the components are added to cells in vitro). The components preferably do not induce inflammation or other adverse effects in vivo. In certain embodiments, the chemical reactions relied upon to break down the biodegradable compounds are enzymatically broken down. For example, the inventive materials may be broken down in part by the hydrolysis of ester bonds.

As used herein, a “biologically active agent” or “active agent,” refers to therapeutic cells, small organic molecules (e.g., hydrophobic and/or hydrophilic drug compounds), peptides, enzymes, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, and vitamins, metals, transition metals, or a combination thereof.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “small organic molecule,” refers to biologically active organic compound which may be either synthesized in the laboratory or isolated or derived from nature, and is composed of carbon, hydrogen, oxygen, nitrogen, sulfur and/or phosphorus, and may have multiple double or triple bonds. In certain embodiments, the small organic molecule is non-peptidic. In certain embodiments, the small organic molecule is non-oligomeric. In certain embodiments, the small organic molecule is a natural product or a natural product-like compound having a partial structure (e.g., a substructure) based on the full structure of a natural product. Exemplary natural products include steroids, penicillins, prostaglandins, venoms, toxins, morphine, paclitaxel, morphine, cocaine, digitalis, quinine, tubocurarine, nicotine, muscarine, artemisinin, cephalosporins, tetracyclines, aminoglycosides, rifamycins, chloramphenicol, asperlicin, lovastatin, ciclosporin, curacin A, eleutherobin, discodermolide, bryostatins, dolostatins, cephalostatins, antibiotic peptides, epibatidine, α-bungarotoxin, tetrodotoxin, teprotide, and neurotoxins from Clostridium botulinum. In certain embodiments, the small molecule has a molecule weight of less than 2000 g/mol. In certain embodiments, the small molecule has a molecular weight of less than 1500 g/mol. In certain embodiments, the small molecule has a molecular weight of less than 1000 g/mol. In certain embodiments, the small molecule has a molecular weight of less than 500 g/mol. Known naturally-occurring small organic molecules include, but are not limited to, penicillin, erythromycin, taxol, cyclosporin, and rapamycin. Known synthetic small molecules include, but are not limited to, ampicillin, methicillin, sulfamethoxazole, and sulfonamides. In certain embodiments, the small organic molecule is a hydrophobic drug (e.g., poorly water soluble). In certain embodiments, the small organic molecule is a hydrophilic drug (e.g., water soluble). In certain embodiments, a small organic molecule is a hydrophobic or hydrophilic drug product approved by the Food and Drug Administration, and as provided in the FDA Code of Federal Regulations (CFR) Title 21, in the FDA Orange Book, or as provided by the FDA Center for Drug Evaluation and Research (CDER).

### BRIEF DESCRIPTION OF THE FIGURES

FIG. 1. Synthetic route and structure of symmetrical 1,3-diglycerides.

FIG. 2. ¹H-NMR spectra of C₁₂ lipid (dodecanoic acid 3-dodecanoyloxy-2-oxo-propyl ester). The C₁₂ lipid's spectra serves as a representative of the remaining lipids.

FIG. 3. Effect of pre-emulsion lipid solution concentration on particle size for a constant 5% PVA concentration. The mean particle diameter for high concentration C₁₂ lipid, as well as C₁₀ and C₈ is statistically significant from its low concentration counterpart (n=3, p<0.05, ±SEM), indicated by asterisks (*).

FIG. 4. Effect of pre-emulsion lipid solution concentration on particle size for a constant 2.5% PVA concentration. Error bars represent ±SEM.

FIG. 5. Effect of varying PVA concentration with constant high pre-emulsion lipid solution concentration. At high concentration (0.02 g/ml), the mean particle diameter of C₁₂ lipid sample decreases with increasing surfactant concentration, and is statistically significant (n=3, p<0.05, ±SEM), denoted by an asterisk (*).

FIG. 6. Effect of varying surfactant concentration with constant low pre-emulsion lipid solution concentration. At low concentration (0.01 g/ml), mean particle diameter for C₈ lipid sample and C₁₀ lipid sample as well as C₁₄ and C₁₆ diameters decrease with increasing surfactant concentration, and is statistically significant (n=3, p<0.05, ±SEM) as indicated by asterisks (*).

FIGS. 7A-7E. Lipid microparticle morphology as a function of lipid chain length. FIG. 7A: C₈ particle morphology showing a smooth surface; FIG. 7B: C₁₀ particle morphology; FIG. 7C: C₁₂ particle morphology; FIG. 7D: C₁₄ particle morphology; and FIG. 7E: C₁₆ particle morphology. Note the increasing porosity with increasing lipid chain length.

FIG. 8. Contact angle of water on lipid surfaces. As expected, surfaces composed of lipids with longer chain length are more hydrophobic compared to those with shorter chain length.

FIG. 9. Zeta potential of lipid microparticles in 1:10 dilutions of PBS:Water. All lipid particle surfaces retain a negative charge.

FIG. 10. Encapsulation efficiency for red nile loaded lipid particles. Encapsulation efficiency results for lipids C₈-C₁₆ for nile red (hydrophobic model drug) show high values of more than 70%.

FIG. 11. Encapsulation efficiency for rhodamine-B loaded lipid particles. Columns marked by asterisk (*) are statistically significant. Encapsulation efficiency of lipids C₁₀-C₁₆ show statistical significance for encapsulation of rhodamine-B model drug, (n=9, p<0.05, ±SEM). No particular trend is observed between encapsulation efficiency and lipid chain length. Lipids show distinct crystallization behavior and smooth morphology in case of shorter chain length lipids (C₈-C₁₀), a possible explanation for high encapsulation efficiency of C₈ compared to longer chain length lipids.

FIG. 12. Nile red release from lipid microparticles. All lipids display slow release over 24 hours (n=3, ±SEM). Release rates increase with increasing lipid chain length.

FIG. 13. Release of rhodamine-B from lipid microparticles. All lipids display burst release (n=3, ±SEM). The observed trends between rhodamine-B release and lipid chain length are unclear.

FIGS. 14A-14J. Model drug distribution within lipid microparticles. FIG. 14A: nile red encapsulated C₈ particles; FIG. 14B: rhodamine-B encapsulated C₈ particles; FIG. 14C: nile red encapsulated C₁₀ particles; FIG. 14D: rhodamine-B encapsulated C₁₀ particles; FIG. 14E: nile red encapsulated C₁₂ particles; FIG. 14F: rhodamine-B encapsulated C₁₂ particles; FIG. 14G: nile red encapsulated C₁₄ particles; FIG. 14H: rhodamine-B encapsulated C₁₄ particles; FIG. 14I: nile red encapsulated C₁₆ particles; FIG. 14J: rhodamine-B encapsulated C₁₆ particles. It is apparent that with increasing lipid chain length the hydrophilic model drug distribution moves toward the surface of the particle, while the hydrophobic model drug remains homogenously distributed.

### DETAILED DESCRIPTION OF CERTAIN EMBODIMENTS OF THE INVENTION

The number of applications for polymeric materials is rapidly growing, leading to a corresponding need for the development of new kinds of biomaterials. Dihydroxyacetone-derived compounds are ideal candidates for this purpose. Dihydroxyacetone (DHA) is a constituent of the glycolysis pathway, and is FDA approved for oral and topical administration, making it an attractive building block for the construction of novel materials (e.g., for example, materials such as particles, films, coatings, micelles, and the like).

Thus, in certain aspects, the present invention provides compounds of Formula I (e.g., an acetylated DHA compound), and pharmaceutically acceptable salts, prodrugs, and/or derivatives thereof.

In certain aspects, the present invention provides pharmaceutical compositions comprising one or more compounds of Formula I, a pharmaceutically acceptable excipient, and optionally, one or more biologically active and/or diagnostic agents.

In certain aspects, the present invention provides inventive materials comprising one or more compounds of Formula I and optionally, one or more biologically active and/or diagnostic agents. Inventive materials include, but are not limited to, particles, films, coatings, micelles, and the like, comprising one or more compounds of Formula I, and optionally, one or more biologically active agents and/or one or more diagnostic agents.

Additionally, the present invention provides pharmaceutical compositions comprising an inventive material, as described herein, and a pharmaceutically acceptable excipient.

Furthermore, the present invention provides methods of making compounds of Formula I and inventive materials.

For example, structural derivatives of DHA, as depicted by Formula II, wherein [symbol] Y, X, R², a, and b, are defined herein, are envisioned as attractive material building blocks for compounds of Formula I. Compounds of Formula I may be synthesized from covalent conjugation of a compound of Formula II (e.g., DHA) with a moiety —(C═Z)—R¹, wherein Z and R¹ are defined herein.

Moreover, the present invention provides methods of using pharmaceutical compositions and inventive materials. For example, in certain embodiments, the inventive materials are solid lipid microparticles (SLM) for drug delivery. In certain embodiments, compounds of Formula I and/or the inventive materials are biodegradable. In certain embodiments, compounds of Formula I and/or the inventive materials are biocompatible. In certain embodiments, upon injestion, compounds of Formula I may biodegrade to their respective components (e.g., by hydrolysis to a compound of Formula II and HO(C═Z)—R¹) through natural metabolic pathways.

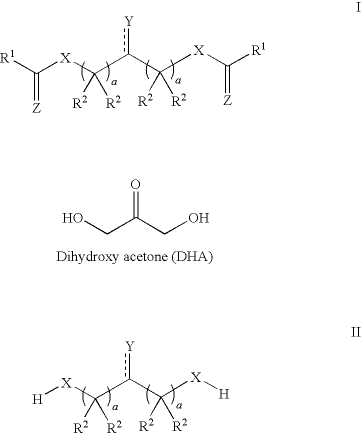

### Compounds of Formula I

The present invention provides compounds of Formula I, or pharmaceutically acceptable salts, prodrugs, or derivatives thereof:

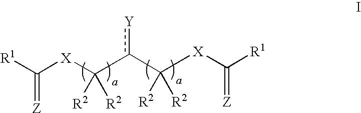

wherein:

[symbol] Y is (═O), —OR^{O}, (═S), —SR^{S}, (═NR^{N}), or —N(R^{N})₂, wherein

R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group;

R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thio protecting group; and

each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

each instance of X is, independently, —O—, —S—, or —N(R^{N})—;

each instance of Z is, independently, O, S, or N(R^{N});

each instance of R¹ is, independently, cyclic or acyclic, substituted or unsubstituted aliphatic; cyclic or acyclic, substituted or unsubstituted heteroaliphatic; substituted or unsubstituted aryl; or substituted or unsubstituted heteroaryl;

each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and

each instance of a is, independently, an integer between 1 to 6, inclusive.

In certain embodiments, a is equal to 1, 2, or 3. In certain embodiments, a is 1. In certain embodiments, a is 2. In certain embodiments, a is 3. In certain embodiments, each instance of a is the same. In certain embodiments, each instance of a is different.

In certain embodiments, each instance of R² is, independently, hydrogen; substituted or unsubstituted C₁₋₆ aliphatic; or substituted or unsubstituted C₁₋₆ heteroaliphatic. In certain embodiments, each instance of R² is hydrogen. In certain embodiments, each instance of R² is the same. In certain embodiments, each instance of R² is different.

In certain embodiments, [symbol] Y is —O. In certain embodiments, [symbol] Y is ═S. In certain embodiments, [symbol] Y is ═N(R^{N}). In certain embodiments, [symbol] Y is ═NH.

In certain embodiments, X is —O—. In certain embodiments, X is —S—. In certain embodiments, X is —N(R^{N})—. In certain embodiments, each instance of X is the same. For example, in certain embodiments, each instance of X is —O—. In certain embodiments, each instance of X is —S—. In certain embodiments, each instance of X is —NH— or —N(R^{N})—. However, in certain embodiments, each instance of X is different.

In certain embodiments, Z is O. In certain embodiments, Z is S. In certain embodiments, Z is N(R^{N}). In certain embodiments, each instance of Z is the same. For example, in certain embodiments, each instance of Z is O. In certain embodiments, each instance of Z is S. In certain embodiments, each instance of Z is NH or N(R^{N}). However, in certain embodiments, each instance of Z is different.

In certain embodiments, the compound of Formula I is of the Formula I-1:

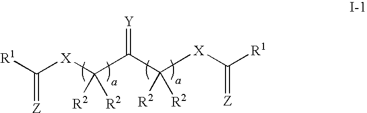

wherein R¹, R², Z, X, and a are as defined above and herein; and

[symbol] Y is ═O, ═S, or ═NR^{N}, wherein R^{N} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group.

In certain embodiments, the compound of Formula I is of the Formula I-2:

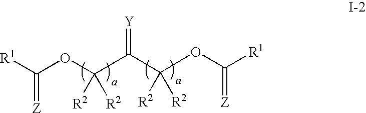

wherein [symbol] Y, Z, R¹, R², and a are as defined above and herein.

In certain embodiments, the compound of Formula I is of the Formula I-3:

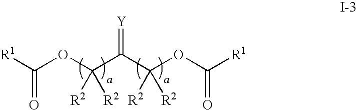

wherein [symbol] Y, R¹, R², and a are as defined above and herein.

In certain embodiments, the compound of Formula I is of the Formula I-4:

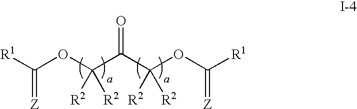

wherein Z, R¹, R², and a are as defined above and herein.

In certain embodiments, the compound of Formula I is of the Formula I-5:

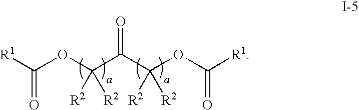

wherein R¹, R², and a are as defined above and herein.

In certain embodiments, the compound of Formula I is of the Formula I-6:

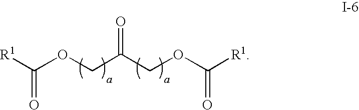

wherein R¹, R², and a are as defined above and herein.

In certain embodiments, the compound of Formula I is of the Formula I-7:

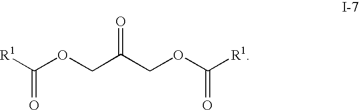

wherein R¹ is as defined above and herein.

However, in certain embodiments, a compound of any of the above Formulae wherein a is 1; R² is hydrogen; and R¹ is acyclic and substituted or unsubstituted C₁₋₃₂ alkyl is specifically excluded. In certain embodiments, a compound of any of the above Formulae wherein a is 1; R² is hydrogen; and R¹ is acyclic and substituted or unsubstituted C₁₋₃₂ alkenyl is specifically excluded. In certain embodiments, a compound of any of the above Formulae wherein a is 1; R² is hydrogen; and R¹ is acyclic and substituted or unsubstituted C₁₋₃₂ alkynyl is specifically excluded.

### Group R¹

As defined generally above, each instance of R¹ is, independently, a cyclic or acyclic, substituted or unsubstituted aliphatic; a cyclic or acyclic, substituted or unsubstituted heteroaliphatic; a substituted or unsubstituted aryl; or a substituted or unsubstituted heteroaryl group.

In certain embodiments, each instance of R¹ is, independently, a cyclic or acyclic, substituted or unsubstituted aliphatic or a cyclic or acyclic, substituted or unsubstituted heteroaliphatic group.

In certain embodiments, each instance of R¹ is, independently, a cyclic or acyclic, substituted or unsubstituted aliphatic group. Aliphatic groups include alkyl, alkenyl and alkynyl groups. In certain embodiments, each instance of R¹ is, independently, a cyclic or acyclic, substituted or unsubstituted alkyl. In certain embodiments, each instance of R¹ is, independently, a cyclic or acyclic, substituted or unsubstituted alkenyl. In certain embodiments, each instance of R¹ is, independently, a cyclic or acyclic, substituted or unsubstituted alkynyl.

In certain embodiments, each instance of R¹ is, independently, a cyclic or acyclic, substituted or unsubstituted heteroaliphatic group. Heteroaliphatic groups include heteroalkyl, heteroalkenyl and heteroalkynyl groups. In certain embodiments, each instance of R¹ is, independently, a cyclic or acyclic, substituted or unsubstituted heteroalkyl. In certain embodiments, each instance of R¹ is, independently, a cyclic or acyclic, substituted or unsubstituted heteroalkenyl. In certain embodiments, each instance of R¹ is, independently, a cyclic or acyclic, substituted or unsubstituted heteroalkynyl.

In certain embodiments, each instance of R¹ is, independently, an even-numbered aliphatic (e.g., alkyl, alkenyl, alkynyl) or heteroaliphatic (e.g., heteroalkyl, heteroalkenyl, heteroalkynyl) group, such as, for example, a C₂, C₄, C₆, C₈, C₁₀, C₁₂, C₁₄, C₁₆, C₁₈, C₂₀, C₂₂, C₂₄, C₂₆, C₂₈, C₃₀ or a C₃₂ group. In certain embodiments, R¹ is, independently, an even-numbered aliphatic or heteroaliphatic group which falls in the range of C₂ to C₃₂, such as a cyclic or acyclic, substituted or unsubstituted C₂₋₃₂ group; cyclic or acyclic, substituted or unsubstituted C₂₋₃₀ group; a cyclic or acyclic, substituted or unsubstituted C₂₋₂₆ group; cyclic or acyclic, substituted or unsubstituted C₂₋₂₀ group; cyclic or acyclic, substituted or unsubstituted C₂₋₁₈ group; cyclic or acyclic, substituted or unsubstituted C₂₋₁₂ group; cyclic or acyclic, substituted or unsubstituted C₂₋₁₀ group; cyclic or acyclic, substituted or unsubstituted C₂₋₆ group; a cyclic or acyclic, substituted or unsubstituted C₆₋₃₀ group; cyclic or acyclic, substituted or unsubstituted C₈₋₃₀ group; cyclic or acyclic, substituted or unsubstituted C₁₀₋₃₀ group; cyclic or acyclic, substituted or unsubstituted C₁₀₋₂₆ group; or a cyclic or acyclic, substituted or unsubstituted C₈₋₁₈ group.

In certain embodiments, each instance of R¹ is, independently, an odd-numbered aliphatic (e.g., alkyl, alkenyl, alkynyl) or heteroaliphatic (e.g., heteroalkyl, heteroalkenyl, heteroalkynyl) group, such as, for example, a C₁, C₃, C₅, C₇, C₉, C₁₁, C₁₃, C₁₅, C₁₇, C₁₉, C₂₁, C₂₃, C₂₅, C₂₇, C₂₉ or C₃₁ group. In certain embodiments, R¹ is, independently, an odd-numbered aliphatic or heteroaliphatic group which falls in the range of C₁ to C₃₁, such as a cyclic or acyclic, substituted or unsubstituted C₁₋₃₁ group; a cyclic or acyclic, substituted or unsubstituted C₁₋₂₇ group; a cyclic or acyclic, substituted or unsubstituted C₁₋₂₁ group; a cyclic or acyclic, substituted or unsubstituted C₁₋₁₉ group; a cyclic or acyclic, substituted or unsubstituted C₁₋₁₅ group; cyclic or acyclic, substituted or unsubstituted C₁₋₁₃ group; a cyclic or acyclic, substituted or unsubstituted C₁₋₉ group; a cyclic or acyclic, substituted or unsubstituted C₁₋₅ group; a cyclic or acyclic, substituted or unsubstituted C₁₋₃ group; a cyclic or acyclic, substituted or unsubstituted C₃₋₂₉ group; a a cyclic or acyclic, substituted or unsubstituted C₅₋₂₅ group; a cyclic or acyclic, substituted or unsubstituted C₇₋₂₁ group; or a cyclic or acyclic, substituted or unsubstituted C₉₋₁₉ group.

In certain embodiments, R¹ is an unsubstituted aliphatic or heteroaliphatic group. In certain embodiments, R¹ is an acyclic aliphatic or heteroaliphatic group. In certain embodiments, R¹ is a substituted aliphatic or heteroaliphatic group. In certain embodiments, R¹ is a cyclic aliphatic or heteroaliphatic group. In certain embodiments, each R¹ group is the same. In certain embodiments, each R¹ group is different.

For example, in certain embodiments, R¹ is a substituted or unsubstituted C₁-C₃₂ alkyl group. In certain embodiments, R¹ is an unsubstituted C₁-C₃₂ alkyl group. In certain embodiments, R¹ is —CH₃, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃, —(CH₂)₁₉CH₃, —(CH₂)₂₀CH₃, —(CH₂)₂₁ CH₃, —(CH₂)₂₂CH₃, —(CH₂)₂₃ CH₃, —(CH₂)₂₄CH₃, —(CH₂)₂₅CH₃, —(CH₂)₂₆CH₃, —(CH₂)₂₇CH₃, —(CH₂)₂₈CH₃, —(CH₂)₂₉CH₃, —(CH₂)₃₀CH₃, or —(CH₂)₃₁CH₃.

However, in certain embodiments, R¹ is not —CH₃. In certain embodiments, R¹ is not —CH₂CH₃. In certain embodiments, R¹ is not —(CH₂)₂CH₃. In certain embodiments, R¹ is not —(CH₂)₃CH₃. In certain embodiments, R¹ is not —(CH₂)₄CH₃. In certain embodiments, R¹ is not —(CH₂)₅CH₃. In certain embodiments, R¹ is not —(CH₂)₆CH₃. In certain embodiments, R¹ is not —(CH₂)₇CH₃. In certain embodiments, R¹ is not —(CH₂)₈CH₃. In certain embodiments, R¹ is not —(CH₂)₉CH₃. In certain embodiments, R¹ is not —(CH₂)₁₀CH₃. In certain embodiments, R¹ is not —(CH₂)₁₁CH₃. In certain embodiments, R¹ is not —(CH₂)₁₂CH₃. In certain embodiments, R¹ is not —(CH₂)₁₃CH₃. In certain embodiments, R¹ is not —(CH₂)₁₄CH₃. In certain embodiments, R¹ is not —(CH₂)₁₅CH₃. In certain embodiments, R¹ is not —(CH₂)₁₆CH₃. In certain embodiments, R¹ is not —(CH₂)₁₇CH₃. In certain embodiments, R¹ is not —(CH₂)₁₈CH₃. In certain embodiments, R¹ is not —(CH₂)₁₉CH₃. In certain embodiments, R¹ is not —(CH₂)₂₀CH₃. In certain embodiments, R¹ is not —(CH₂)₂₁CH₃. In certain embodiments, R¹ is not —(CH₂)₂₂CH₃. In certain embodiments, R¹ is not —(CH₂)₂₃CH₃. In certain embodiments, R¹ is not —(CH₂)₂₄CH₃. In certain embodiments, R¹ is not —(CH₂)₂₅CH₃. In certain embodiments, R¹ is not —(CH₂)₂₆CH₃. In certain embodiments, R¹ is not —(CH₂)₂₇CH₃. In certain embodiments, R¹ is not —(CH₂)₂₈CH₃. In certain embodiments, R¹ is not —(CH₂)₂₉CH₃. In certain embodiments, R¹ is not —(CH₂)₃₀CH₃. In certain embodiments, R¹ is not —(CH₂)₃₁CH₃.

Aliphatic and heteroaliphatic groups also include fatty acid substituents. In certain embodiments, R¹ is a substituent of a C₂-C₃₂ fatty acid, wherein a fatty acid refers to a C₂-C₃₂-substituted carboxylic acid having the Formula HO—(C═O)—R¹, and R¹ is an aliphatic or heteroaliphatic group. Exemplary fatty acids include, but are not limited to, saturated fatty acids, monoenoic fatty acids, polyunsaturated fatty acids, methyl-branched fatty acids, ring-containing fatty acids, methoxy fatty acids, thia fatty acids, keto fatty acids, and oxo fatty acids.

In certain embodiments, R¹ is a substituent of a saturated fatty acid. Exemplary saturated fatty acids include, but are not limited to, ethanoic acid; propanoic acid; butanoic acid; pentanoic acid; hexanoic acid (caproic); heptanoic acid; octanoic acid (caprylic); nonanoic acid; decanoic acid (capric); undecanoic acid; dodecanoic (lauric); tridecanoic acid; tetradecanoic acid (myristic); pentadecanoic acid; hexadecanoic acid (palmitic); heptadecanoic acid; octadecanoic acid (stearic); nonadecanoic acid; eicosanoic acid (arachidic); heneicosanoic acid; docosanoic acid (behenic); tricosanoic acid; etracosanoic acid (lignoceric); pentacosanoic acid; hexacosanoic acid; heptacosanoic acid; octacosanoic acid; nonacosanoic acid; triacontanoic acid; hentriacontanoic acid; and dotriacontanoic acid. However, in certain embodiments, R¹ is not a substituent of a saturated fatty acid.

In certain embodiments, R¹ is a substituent of a monoenoic fatty acid. Exemplary monoenoic fatty acids include, but are not limited to, cis-9-hexadecenoic acid (palmitoleic); cis-6-octadecenoic acid (petroselinic); cis-9-octadecenoic acid (oleic); cis-11-octadecenoic acid (cis-vaccenic); trans-11-octadecenoic acid (trans-vaccenic acid), cis-13-docosenoic acid (erucic); and cis-15-tetracosenoic acid (nervonic). However, in certain embodiments, R¹ is not a substituent of a monoenoic fatty acid.

In certain embodiments, R¹ is a substituent of a polyunsaturated fatty acid. Exemplary polyunsaturated fatty acids include, but are not limited to, 9,12-octadecadienoic acid (linoleic); 6,9,12-octadecatrienoic acid (γ-linolenic); 9,12,15-octadecatrienoic acid (α-linolenic); 5,8,11,14-eicosatetraenoic acid (arachidonic); 5,8,11,14,17-eicosapentaenoic acid (20:5(n-3) or EPA); 4,7,10,13,16,19-docosahexaenoic acid (22:6(n-3) or DHA); 11,14,17-eicosatrienoic acid; 6,9,12,15-octadecatetraenoic acid (stearidonic); 3,6,9,12,15-octadecapentaenoic acid; 8,11,14,17-eicosatetraenoic acid; 7,10,13,16,19-docosapentaenoic acid; 5,8,11-eicosatrienoic acid; 9,12-hexadecadienoic acid; 9-cis,11-trans-octadecadienoic acid; 2-trans,4-trans-hexadienoic acid (sorbic), trans-7,cis-9-octadecadienoic acid; and 6,9,12-hexadecatrienoic acid. However, in certain embodiments, R¹ is not a substituent of a polyunsaturated fatty acid.

In certain embodiments, R¹ is a substituent of a methyl-branched fatty acid. Exemplary methyl-branched fatty acids include, but are not limited to, iso-methyl branched fatty acids, in which the carbon chain has the branch point on the penultimate carbon (one from the end); anteiso-methyl-branched fatty acids, in which the carbon chain has the branch point on the ante-penultimate carbon atom (two from the end); neo fatty acids, which have either a terminal tertiary butyl group, or two iso-methyl groups (e.g., 3,13-dimethyltetradecanoic acid or ‘neopalmitic acid’); isoprenoid fatty acids (e.g., 3,7,11,15-tetramethylhexadec-trans-2-en-1-ol; 2,6-dimethylheptanoic acid; 5,9,13,17-tetramethyloctadecanoic acids; 3,7,11,15-tetramethylhexadecanoic acid (phytanic); 2,6,10,14-tetramethylpentadecanoic acid (pristanic); 4,8,12-trimethyltridecanoic acid; 10-R-methyloctadecanoic acid (tuberculostearic acid); 10-methylhexadecanoic acid; 11-methyloctadecanoic acid; 12-methylhexadecanoic acid; 14-methyloctadecanoic acid; 8-methylhexadecanoic acid; 10-methylhexadecanoic acid; 9-methylheptadecanoic acid; 11-methylnonadecanoic acid; 12-methyleicosanoic acid; 14-methyldocosanoic acid; and 16-methyltetracosanoic acid); unsaturated methyl-branched fatty acids (e.g., 13-methyltetradec-4-enoic acid, 14-methylhexadec-6-enoic acid, 14-methylpentadec-6-enoic acid; 17-methyloctadec-8-enoic acid; 16-methyl-cis-9-octadecenoic acid; and 16-methyl-cis-9,cis-12-octadecadienoic acid). However, in certain embodiments, R¹ is not a substituent of a methyl-branched fatty acid.

In certain embodiments, R¹ is a substituent of a ring-containing fatty acid. Exemplary ring-containing fatty acids include, but are not limited to, cis-11,12-methylene-octadecanoic acid; cis-9,10-methylene-octadecanoic acid (dihydrosterculic acid); 3-hydroxy-lactobacillic acid; 7-methyl-cis-9,10-methylene-octadecanoic acid; 9,10-methylene-5-hexadecenoic acid; 11,12-methylene-5-octadecenoic acid; 11-cyclohexylundecanoic acid; 13-cycloheptyltridecanoic acids; 9,10-methylene-heptadec-9-enoic acid (malvalic acid); 2-hydroxysterculic acid, 9,10-methylene-octadec-9-en-17-ynoic acid (sterculynic acid); 11-cyclopent-2-enyl-undecanoic acid (hydnocarpic), 13-cyclopent-2-enyl-tridecanoic acid (chaulmoogric); and 13-cyclopent-2-enyltridec-6-enoic acid (gorlic acid); 15-cyclopent-2-enylpentadec-9-enoic acid (hormelic); 13-cyclopent-2-enyltridec-4-enoic acid; 11-cyclopentylundecanoic acid; cyclopent-2-enylcarboxylic acid (aleprolic); 13-phenyltridecanoic acid; and 2-hydroxy-11,12-methylene-docos-5-enoate acid. However, in certain embodiments, R¹ is not a substituent of a ring-containing fatty acid.

In certain embodiments, R¹ is a substituent of a hydroxy fatty acid. Exemplary hydroxy fatty acids include, but are not limited to, 2-(D)-hydroxy fatty acids (e.g., 2-hydroxydocasanoic acid, 2-hydroxytetracosanoic acid (cerebronic acid), 2-hydroxy-15-tetracosenoic acid (hydroxynervonic acid)); 2,3-dihydroxy-long-chain fatty acids (e.g., 2-hydroxy-phytanic acid); 3-hydroxy-fatty acids (e.g., mycolic acids; (β-hydroxybutoic acid); 3-hydroxydicarboxylic acids (e.g., 3-hydroxy-pristanic acid); hydroxy-keto-fatty acids; hydroxyl fatty acid components of beeswax (e.g., 15-hydroxy-hexadecanoic acid; 17-hydroxy-octadecanoic acids; and other homologues and (ω-2)-hydroxy-isomers; royal jelly which contains a number of mono- and dihydroxy fatty acids (C₈ to C₁₄); 9,10,16-trihydroxy-hexadecanoic acid (aleuritic) 6-hydroxy-tetradecanoic acid); fatty acids found in seed oils (e.g., 2-hydroxy-octadeca-9,12,15-trienoate; 2-hydroxy-oleic and linoleic acids; 2-hydroxy-sterculic acid; ricinoleic acid (D-(−)12-hydroxy-octadec-cis-9-enoic acid); conjugated dienoic fatty acids such as 9-hydroxy-octadeca-trans-10,trans-12-dienoic (dimorphecolic); 9-hydroxy-octadeca-trans-10,cis-12-dienoic acid; and 13-hydroxy octadeca-cis-9,trans-11-dienoic (coriolic) acid); conjugated diynoic fatty acids such as ximenynolic (8-hydroxy,9a,11t) and isanolic (8-hydroxy,9a,11a,17e) acids); the fatty acids of cutins (e.g., ω-monohydroxy acids, 9(or 10),16-dihydroxy-hexadecanoic acid (and analogous C₁₈ acids)), 9,10,18-trihydroxy-octadecanoic acid, and related fatty acids with epoxy or keto groups in central positions, e.g. 9,10-epoxy,18-hydroxy-octadecanoate); 10-hydroxy-stearic acid; epoxy fatty acids (e.g., (+)-vernolic acid; cis-12,13-epoxy-octadec-cis-9-enoic acid; cis-9,10-epoxy-octadec-cis-12-enoic ('coronaric') acid; cis-15,16-epoxy-octadeca-cis-9,12-dienoic acid; cis-9,10-epoxy-octadecanoic acid; and 10,13-epoxy-11,12-dimethyloctadeca-10,12-dienoic acid); furanoid fatty acids (e.g., 12,15-epoxy-13,14-dimethyleicosa-12,14-dienoic acid; 12,15-epoxy-13-methyleicosa-12,14-dienoic acid; and 3-carboxy-4-methyl-5-propyl-2-furanpropanoic acid). However, in certain embodiments, R¹ is not a substituent of a hydroxy fatty acid.

In certain embodiments, R¹ is a substituent of a methoxy fatty acid. Exemplary methoxy fatty acids include, but are not limited to, 7-methoxy,9-methyl-hexadeca-4t,8t-dienoic acid; 2-methoxy-5-hexadecenoic acid; 2-methoxy-6-hexadecenoic acid; and 2-methoxy hexadecanoic acid.). However, in certain embodiments, R¹ is not a substituent of a methoxy fatty acid.

In certain embodiments, R¹ is a substituent of a thia fatty acid. Exemplary thia fatty acids (sulfur-containing fatty acids) include, but are not limited to 3-thia fatty acids (e.g., dodeca thia acetic acid CH₃—(CH₂)₁₁—S—CH₂—COOH, tetradeca thia acetic acid CH₃—(CH₂)₁₃—S—CH₂—COOH), and 4-thia fatty acids. However, in certain embodiments, R¹ is not a substituent of a thia fatty acid.

In certain embodiments, R¹ is a substituent of a keto fatty acid. Exemplary keto fatty acids include, but are not limited to, 9-keto-2t-decenoic acid. However, in certain embodiments, R¹ is not a substituent of a thia fatty acid.

In certain embodiments, R¹ is a substituent of an oxo fatty acid. Exemplary oxo fatty acids include, but are not limited to, traumatin (12-oxo-9Z-dodecenoic acid); 9-hydroxy-traumatin; 11-hydroxy-traumatin; and 13-oxo-9Z-11E-tridecadienoic acid. However, in certain embodiments, R¹ is not a substituent of an oxo fatty acid.

### Exemplary Compounds

In certain embodiments, a compound of Formula I, or subset thereof, is selected from any of the following compounds:

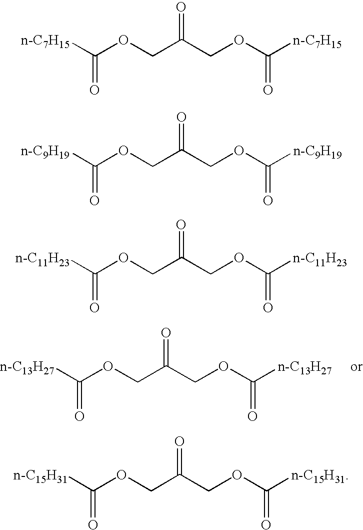

### Inventive Materials

In certain embodiments, the present invention provides a material comprising one or more compounds of Formula I. In certain embodiments, the present invention provides a material comprising one or more compounds of Formula I, and one or more biologically active agents. In certain embodiments, the present invention provides a material comprising one or more compounds of Formula I, and one or more diagnostic agents. It should be understood that a “material,” as used herein, refers to an organic material capable of biodegradation. Materials of the present invention may take on any kind of form, shape, or consistency, such as, for example, a tablet, a particle (e.g., a microparticle, a nanoparticle, a picoparticle), a film, a sheet, a coating, a micelle, a liposome, a rod, a tube, a spheroid, a cone, a composite, a matrix (e.g., lipid matrices), a liquid-like consistency, a lotion, a cream, a gel, a hydrogel, an elastomer, a plastic consistency, a rubber-like consistency, a granular or powdery consistency, an amorphous form, a crystalline form, and the like. In certain aspects, the inventive material is also biocompatible.

In certain embodiments, the inventive material has an in-vivo half life of between 0.1 hour to 5 years. In certain embodiments, the inventive material has an in-vivo half life of at least about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours 7 hours, 8 hours, 9 hours, 10 hours, 11 hours, 12 hours, 24 hours, 2 days, 3 days, 4 days, 5 days, 6 days, 1 week, 2 weeks, 3 weeks, 4 weeks, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, or at least about 5 years.

### (i) Biologically Active Agents

In certain embodiments, the present invention provides a material comprising one or more compounds of Formula I, and one or more biologically active agents.

In certain embodiments, the inventive material comprises one or more compounds of Formula I and a therapeutically effective amount of one or more biologically active agents. As used herein, a “therapeutically effective amount,” refers to the amount or concentration of a biologically active agent present in a pharmaceutical composition or inventive material of the presently claimed invention, that, when administered to a subject, is effective to at least partially treat a condition from which the subject is suffering.

As used herein, a “biologically active agent” refers to therapeutic cells, small organic molecules, amino acids, dipeptides, peptides, polypeptides, proteins, enzymes, carbohydrates, monosaccharides, oligosaccharides, polysaccharides, nucleoproteins, mucoproteins, lipoproteins, small molecules linked to proteins, glycoproteins, steroids, nucleic acids, DNAs, RNAs, nucleotides, nucleosides, oligonucleotides, antisense oligonucleotides, lipids, hormones, vitamins, metals, transition metals, organometals, or combinations thereof.

Additionally, in certain embodiments, a “biologically active agent” refers to insecticides, microbial pesticides, herbicides, botanical insecticides, and other pest control chemicals, as well as pheromone lures, alarming pheromones, sex pheromones, and pheromone insect traps.

In certain embodiments, the biologically active agent is a small organic molecule. In certain embodiments, the small organic molecule has a molecular weight of less than about 2000 g/mol (grams per mol), 1500 g/mol, 1000 g/mol, 500 g/mol, or of less than about 300 g/mol. In certain embodiments, the biologically active agent a hydrophilic (i.e., water soluble) organic molecule. In certain embodiments, the biologically active agent a hydrophobic (i.e., poorly water-soluble) organic molecule. Exemplary small organic molecules include paclitaxel, docetaxel, doxorubicin, cisplatin, carboplatin, 5-FU, etoposide, camptothecin, hormones and steroids (e.g., testosterone, estrogen, estradiol, triamcinolone acetonide, hydrocortisone, dexamethasone, prednisolone, betamethasone), cyclosporines, hydrophobic peptides, polypeptides, proteins, nucleotides, oligonucleotides, nucleases, calcitonin, insulin, erthropoietin, prostaglandins, and the like. Small organic molecules also include pesticides, herbicides, fungicides, and organic pesticides such as acephate, acetamiprid, thiamethoxam, lamba cyhalothrin, sulfosulfuron, metsulfuron methyl, hexaconazole, tricyclazole, imidacloprid, thiamethoxam, metalaxyl and alpha cypermethin.

Biologically active agents also include any substance used as a medicament for the treatment, prevention, delay, reduction or amelioration of a disease, condition, or disorder, (or infestation), and also refers to a substance that is useful for therapy, including prophylactic treatment. Additionally, a biologically active agent includes any substance that increases the effect or effectiveness of another substance, for example, by enhancing potency or reducing adverse effects of another biologically active agent.

In certain embodiments, the biologically active agent is an anti-anxiety agent, an anti-convulsant agent, an anti-depressant agent, an anti-psychotic agent, a sedative, a stimulant, an analgesic, an antacid, an antiarrhythmic, an antibacterial agent, an antifungal agent, an an antibiotic, an anticoagulant, a thrombolytic agent, an anticonvulsant, an antidiarrheal agent, antiemetic agent, antifungal agent, an antihistamine, an antihypertensive agent, an anti-inflammatory agent, an antineoplastic agent, an antipsychotic agent, an antipyretic (chemotherapeutic) agent, an antiviral agent (e.g., protease inhibitors for HIV; acyclovir for herpes virus, etc.), a barbiturate, a beta-blocker, a bronchodilator, a corticosteroid, a steroid, a diuretic, an anti-inflammatory agent, a hormone, a vaccine, a hypoglycemic, an immunosuppressive, a muscle relaxant, a vasoconstrictors, a cytokine such as interleukins, a colony stimulating factor, tumor necrosis factor, an interferon (e.g., alpha, beta, gamma), a non-steroidal anti-inflammatory drug (NSAIDs), an anesthetic, a steroidal agent, an antigen, an antibody, a decongestant, an antihypertensive, a birth control agent, a anti-acne agent; a bronchodilator, a progestational agent, a anti-cholinergic agent, an analgesic, an anti-depressant, a (β-adrenergic blocking agent, a cardiovascular active agent, a vasoactive agent, a nutritional agent, and the like.

In certain embodiments, the biologically active agent is a prophylactic agent. Prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, and cell extracts. Prophylactic agents may be combined with interleukins, interferon, cytokines, and adjuvants such as cholera toxin, alum, Freund's adjuvant, etc. Prophylactic agents include antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In certain embodiments, one or more biologically active agents are conjugated to one or more compounds of Formula I. In certain embodiments, a biologically active agent is non-covalently conjugated/associated to a compound of Formula I. Non-covalent interactions include, but are not limited to, hydrogen bonding, van der Waals interactions, hydrophobic interactions, magnetic interactions, and electrostatic interactions.

In certain embodiments, the biologically active agent is a “prodrug” when non-covalently conjugated with a compound of Formula I, and is administered to a subject in an inactive (or significantly less active) form. Once administered, the associated prodrug is metabolised in vivo to a more active form.

However, in certain embodiments, the biologically active agent is as active when non-covalently conjugated to a compound of Formula I as compared to the free biologically active agent (i.e., not non-covalently conjugated).

In certain aspects, the inventive material is biodegradable. In certain aspects, the inventive material is biocompatible. In certain embodiments, the inventive material, upon biodegrading, releases one or more biologically active agents.

In certain embodiments, the inventive material, upon biodegrading, degrades to one or more biocompatible products, and releases one or more biologically active agents. In certain embodiments, the inventive material, upon biodegrading, degrades to one or more fatty acids, and releases one or more biologically active agents. In certain embodiments, the inventive material, upon biodegrading, degrades to one or more biocompatible fatty acids, and releases one or more biologically active agents. In certain embodiments, the inventive material, upon biodegrading, degrades to one or more biocompatible fatty acids and one or more compounds of Formula I, and releases one or more biologically active agents. In certain embodiments, the inventive material, upon biodegrading, degrades to one or more biocompatible fatty acids and dihydroxyacetone (DHA), and releases one or more biologically active agents.

### (ii) Diagnostic Agents

In certain embodiments, the present invention provides a material comprising one or more compounds of Formula I and one or more diagnostic agents. Diagnostic agents include, but are not limited to, metals (e.g., metals, organometals, and transition metals such as gadolinium chelates, iron, magnesium, manganese, copper, chromium, and the like); radiopharmaceuticals and commercially available imaging or contrast agents used in positron emissions tomography (PET), computer assisted tomography (CAT), single photon emission computerized tomography, x-ray, CT scan, fluoroscopy, and magnetic resonance imaging (MRI); (e.g., iodine-based materials, TECHNESCAN® PYP®, MPI® PYROPHOSPHATE, CIS-PYRO®, PHOSPHOTEC®, ULTRATAG® RBC, PULMOLITE®, MACROTEC®, MPI® MAA®, MPI® MDP®, OSTEOLITE®, MPI® DTPA®, An-DTPA®, MPI® DMSA, CARDIOLITE®, MIRALUMA®).

In certain embodiments, one or more diagnostic agents are conjugated to one or more compounds of Formula I. In certain embodiments, a diagnostic agent is non-covalently conjugated/associated to a compound of Formula I. Non-covalent interactions include, but are not limited to, hydrogen bonding, van der Waals interactions, hydrophobic interactions, magnetic interactions, and electrostatic interactions.

In certain aspects, the inventive material is biodegradable. In certain aspects, the inventive material is biocompatible. In certain embodiments, the inventive material, upon biodegrading, releases one or more diagnostic agents.

In certain embodiments, the inventive material, upon biodegrading, degrades to one or more biocompatible products, and releases one or more diagnostic agents. In certain embodiments, the inventive material, upon biodegrading, degrades to one or more fatty acids, and releases one or more diagnostic agents. In certain embodiments, the inventive material, upon biodegrading, degrades to one or more biocompatible fatty acids, and releases one or more diagnostic agents. In certain embodiments, the inventive material, upon biodegrading, degrades to one or more biocompatible fatty acids and one or more compounds of Formula I, and releases one or more diagnostic agents. In certain embodiments, the inventive material, upon biodegrading, degrades to one or more biocompatible fatty acids and dihydroxyacetone (DHA), and releases one or more diagnostic agents.

(iii) Personal Care Management

In certain embodiments, the present invention provides a material comprising one or more compounds of Formula I useful for improving the appearance of a subject. In certain embodiments, the present invention provides a material useful for improving a subject's appearance comprising one or more compounds of Formula I and one or more biologically active agents.

In certain embodiments, the material is included in a personal care composition or Formulation, such as, for example, a topical composition (e.g., cosmetic, makeup, shampoo, soap, lotion, cream, toothpaste, mouthwash, deodorant, antipersperant, perfume, and the like) and/or an injectable composition (e.g, to inject into the lips to make them more voluptuous). In the context of personal care management, the material of the present invention, or the composition or formulation comprising the material, may take on any form, shape, or consistency, such as, for example, a liquid-like consistency, such as a lotion, a cream, a gel, a spray, an aerosol, or a solid-like consistency, such as a hydrogel, an elastomer, a powder, a particle (e.g., a microparticle, a nanoparticle, a picoparticle), or a composite, which will aid in the application or injection of a material, such as, for example, to the subject's hair, skin, teeth, eyes, lips, etc.

### (iv) Microparticles

A compound of Formula I of the present invention may be used to form drug delivery devices. For example, a compound of Formula I may be used to encapsulate or embed one or more biologically active agents, as defined herein, in the microparticle to provide an inventive material. In certain embodiments, the microparticle is a solid lipid microparticle (SLM).

In certain embodiments, the diameter of the microparticles ranges from between about 500 nm to about 50 micrometers, from about 1 micrometer to about 20 micrometers, from about 1 micrometer to about 10 micrometers, or from about 1 micrometer to about 5 micrometers.

The inventive materials have several properties that make them particularly suitable in the preparation of drug delivery devices, such as the ability of the compound of Formula I to non-covalently complex biologically active agents. In certain embodiments, a compound of Formula I is used to form microparticles containing the biologically active agent to be delivered. These microparticles may include other materials such as proteins, carbohydrates, synthetic polymers (e.g., PEG, PLGA), and natural polymers.

### (v) Micelles and Liposomes

An inventive material may be used to prepare micelles or liposomes. Many techniques for preparing micelles and liposomes are known in the art, and any method may be used with the inventive lipids to make micelles and liposomes. In addition, any agent including polynucleotides, small molecules, proteins, peptides, metals, organometallic compounds, etc. may be included in a micelle or liposome. Micelles and liposomes are particularly useful in delivering hydrophobic biologically active agents such as hydrophobic small molecules.

### Methods of Making Compounds of Formula I

In another aspect, the present invention provides a method of making a compound of Formula I, the method comprising the steps of:

(i) providing a compound of Formula II:

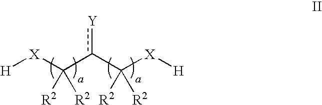

wherein:

[symbol] Y is (═O), —OR^{O}, (═S), —SR^{S}, (═NR^{N}), or —N(R^{N})₂, wherein R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl-protecting group; R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thio protecting group; and each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

each instance of X is, independently, O, S, or N(R^{N});

each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and

each instance of a is, independently, an integer between 1 to 6, inclusive;

(ii) providing one or more compounds of Formula III:

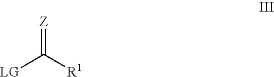

wherein:

Z is, independently, O, S, or N(R^{N}), wherein R^{N} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;

R¹ is cyclic or acyclic, substituted or unsubstituted aliphatic; cyclic or acyclic, substituted or unsubstituted heteroaliphatic; substituted or unsubstituted aryl; or substituted or unsubstituted heteroaryl; and

LG is a suitable leaving group;

(iii) mixing one or more compounds of Formula II with one or more compounds of Formula III to provide a compound of Formula I.

A “suitable leaving group,” or the variable “LG,” as used herein, refers to a charged or uncharged atom that departs during a substitution or displacement reaction. Leaving groups are well known in the art, and include those described in detail in March's Advanced Organic Chemistry: Reactions, Mechanisms, and Structure, M. B. Smith and J. March, 5^{th} Edition, John Wiley & Sons, 2001, the entirety of which is incorporated herein by reference. Exemplary leaving groups include halogens (e.g., bromo, chloro, iodo), activated hydroxyl groups, alkoxy groups, thioalkoxy groups, and the like. Exemplary activated hydroxyl groups include acylated hydroxyl groups, sulfonylated hydroxyl groups (e.g., O-trifluoromethylsulfonyl (-OTf), O-tolylsulfonyl (-OTs), O-methanesulfonyl (-OMs), O-(4-nitrophenylsulfonyl) (-ONos), O-(2-nitrophenylsulfonyl) (-ONs)), and the like. In certain embodiments, LG is —OR^{B}, —OC(O)R^{B}, —OC(O)OR^{B}, —OS(O)R^{B}, —OS(O)₂R^{B}, wherein each R^{B} is cyclic or acylic, substituted or unsubstituted aliphatic group; cyclic or acylic, substituted or unsubstituted heteroaliphatic group; substituted or unsubstituted aryl group; or substituted or unsubstituted heteroaryl group.

In certain embodiments, the above step (iii) further comprises a suitable base. Suitable bases include, but are not limited to, sodium hydroxide, sodium bicarbonate, sodium carbonate, potassium carbonate, potassium hydroxide, lithium hydroxide, calcium oxide, calcium hydroxide, calcium carbonate, magnesium oxide, magnesium hydroxide, cesium carbonate, cesium hydroxide, barium oxide, barium hydroxide, ammonium hydroxide, ammonium chloride, tetrabutylammonium hydroxide, benzyltrimethylammonium hydroxide, triethylbenzylammonium hydroxide, 1,1,3,3-tetramethylguanidine, 1,8-diazabicyclo[5.4.0]undec-7-ene (DBU), N-methylmorpholine, diisopropylethylamine (DIPEA), tetramethylethylenediamine (TMEDA), pyridine (Py), imidazole, 1,4-diazabicyclo[2.2.2]octane (DABCO), N,N-dimethylamino pyridine (DMAP), or triethylamine (NEt₃).

In certain embodiments, the mixing refers to adding one or more compounds of Formula III to a compound of Formula II. In certain embodiments, the mixing refers to adding a compound of Formula II to one or more compounds of Formula III. In certain embodiments, the above step (iii) provides adding a compound of Formula II to one or more compounds of Formula III, followed by the addition of a suitable base, to provide a compound of Formula I. In certain embodiments, the above step (iii) provides adding one or more compounds of Formula III to a compound of Formula II, followed by the addition of a suitable base, to provide a compound of Formula I. In certain embodiments, adding or addition refers to dropwise addition.

In certain embodiments, the above step (iii) is performed in a suitable solvent. Suitable solvents include, but are not limited to, hydrocarbons, halogenated hydrocarbons, esters, ethers, aromatic solvents, polar aprotic solvents, or mixtures thereof. In other embodiments, the solvent is hexanes, pentanes, heptanes, cyclohexane, ethyl acetate, isopropyl acetate, diethylether, tetrahydrofuran, dioxane, methyl tert-butyl ether, toluene, benzene, xylenes, dichloromethane, dichloroethane, chloroform, or a mixture thereof.

In other embodiments, the above step (iii) is conducted with heating (i.e., above room temperature). Alternatively, the above step (iii) is conducted with cooling (i.e., below room temperature).

### Methods of Making Inventive Materials

In certain embodiments, the inventive material is a particle (for example, a microparticle, a nanoparticle, or a picoparticle).

The inventive particles may be prepared using any method known in this art. These include, but are not limited to, spray drying, single and double emulsion solvent evaporation, solvent extraction, phase separation, simple and complex coacervation, and other methods well known to those of ordinary skill in the art. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

If the particles prepared by any of the above methods have a size range outside of the desired range, the particles can be sized, for example, using a sieve. The particle may also be coated. In certain embodiments, the particles are coated with a targeting agent. In other embodiments, the particles are coated to achieve desirable surface properties (e.g., a particular charge).

### (i) Liposomes

In certain embodiments, inventive materials are liposomes.

In certain embodiments, liposomes (lipid vesicles) are formed through spontaneous assembly. In other embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these particles have formed, reducing the size of the particle can be modified through input of sonic energy (sonication) or mechanical energy (extrusion). See Walde, P. “Preparation of Vesicles (Liposomes)” In Encyclopedia of Nanoscience and Nanotechnology; Nalwa, H. S. Ed. American Scientific Publishers: Los Angeles, 2004; Vol. 9, pp. 43-79; Szoka et al. “Comparative Properties and Methods of Preparation of Lipid Vesicles (Liposomes)” Ann. Rev. Biophys. Bioeng. 9:467-508, 1980; each of which is incorporated herein. The preparation of liposomes involves preparing the inventive material for hydration, hydrating the inventive material with agitation, and sizing the vesicles to achieve a homogenous distribution of liposomes. The inventive material is first dissolved in an organic solvent to assure a homogeneous mixture of lipids. The solvent is then removed to form a film. This film is thoroughly dried to remove residual organic solvent by placing the vial or flask on a vacuum pump overnight. Hydration of the film/cake is accomplished by adding an aqueous medium to the container of dry material and agitating the mixture. Disruption of LMV suspensions using sonic energy typically produces small unilamellar vesicles (SUV) with diameters in the range of 15-50 nm. Extrusion is a technique in which a suspension of the material is forced through a polycarbonate filter with a defined pore size to yield particles having a diameter near the pore size of the filter used. Extrusion through filters with 100 nm pores typically yields large, unilamellar vesicles (LUV) with a mean diameter of 120-140 nm. In certain embodiments, the inventive material spontaneously self assembles around certain molecules, such as DNA and RNA, to form liposomes.

The following scientific papers described other methods for preparing liposomes and micelles: Narang et al. “Cationic Lipids with Increased DNA Binding Affinity for Nonviral Gene Transfer in Dividing and Nondividing Cells” Bioconjugate Chem. 16:156-68, 2005; Hofland et al. “Formation of stable cationic lipid/DNA complexes for gene transfer” Proc. Natl. Acad. Sci. USA 93:7305-7309, July 1996; Byk et al. “Synthesis, Activity, and Structure—Activity Relationship Studies of Novel Cationic Lipids for DNA Transfer” J. Med. Chem. 41(2):224-235, 1998; Wu et al. “Cationic Lipid Polymerization as a Novel Approach for Constructing New DNA Delivery Agents” Bioconjugate Chem. 12:251-57, 2001; Lukyanov et al. “Micelles from lipid derivatives of water-soluble polymers as delivery systems for poorly soluble drugs” Advanced Drug Delivery Reviews 56:1273-1289, 2004; Tranchant et al. “Physicochemical optimisation of plasmid delivery by cationic lipids” J. Gene Med. 6:S24-S35, 2004; van Balen et al. “Liposome/Water Lipophilicity: Methods, Information Content, and Pharmaceutical Applications” Medicinal Research Rev. 24(3):299-324, 2004; each of which is incorporated herein by reference.

### (ii) Particles

In other aspects, the present invention provides a method of making an inventive material, the method comprising:

(i) providing one or more compounds of the Formula I;

(ii) providing one or more biologically active agents or diagnostic agents, as described above and herein;

(iii) adding one or more compounds and one or more biologically active agents or diagnostic agents to a suitable solution to provide a mixture; and

(iv) stirring the mixture of one or more compounds and one or more biologically active agents or diagnostic agents with evaporation of the suitable solution to provide the material.

In certain embodiments, the inventive material formed by the above method is a particle (e.g., a microparticle, a nanoparticle, a picoparticle, and the like).

In certain embodiments, the above step (iii) further includes:

(iii) adding one or more compounds and one or more biologically active agents or diagnostic agents to a suitable solution to provide a first mixture, and transferring this first mixture to an aqueous medium to provide a second mixture.

In certain embodiments, the above step (iv) further provides:

(iv) stirring the mixture of one or more compounds and one or more biologically active agents with evaporation of the suitable solvent to provide a material, wherein one or more biologically active agents is non-covalently associated (i.e., not covalently attached to) to one or more compounds.

In certain embodiments, the aqueous medium is water. In certain embodiments, the aqueous medium comprises water and a water-soluble polymer. In certain embodiments, the aqueous medium comprises at least about 0.5%, 1%, 2%, 3%, 4%, 5%, 6%, 7%, 8%, 9%, 10%, 15%, or 20%, of a water-soluble polymer. Exemplary water-soluble polymers include, but are not limited to, polyvinyl alcohol (PVA), polysaccharides (e.g., dextran, cellulose, glycogen), polyethylene glycol, poly[N-(2-hydroxypropyl)methacrylamide (pHPMA), and polyhydroxymethacrylate (pHEMA).

In certain embodiments, the above step (ii) or (iii) further includes one or more porogens, dispersing agents, emulsifying agents, surfactants (e.g., sodium dodecyl sulphate (SDS), phospholipids, monoglycerides, diglycerides, polysorbate), salts (e.g., CaCl₂, NaCl, KCl), or cyclic sugars (e.g., maltitol, lactitol, isomalt, allose, altrose, glucose, mannose, gulose, idose, galactose, talose, ribose, arabinaose, xylose, lyxose, maltose, cellobiose, sucrose, trehalose, lactose, amylose, amylopectin, glycogen, cellulose, fructofuranose, glucopyranose, sorbose, rhaminose, tagatose, apiose, deoxyribose, ribofructose, 1,3,6-tri-O-galloyl-β-D-glucopyranose (tannic acid); amino-containing cyclic sugars (e.g., N-acetyl glucoseamine (sialic acid), glucoseamine); amide-containing cyclic sugars (e.g., glucoronamide); carboxyl containing sugars (e.g., galacturonic acid).

In certain embodiments, the above step (iii) further comprises transferring the first mixture to an aqueous medium, wherein the aqueous medium is vigorously stirring. In certain embodiments, the above step (iii) further comprises transferring droplets of the first mixture to an aqueous medium, wherein the aqueous medium is vigorously stirring. In certain embodiments, the above step (iii) further comprises transferring the first mixture to an aqueous medium by pipet.

In certain embodiments, the vigorous stirring is a vigorous vortex. In certain embodiments, the mixture is vortexed at speeds of at least about 10,000 rpm (revolutions per minute), at least about 9,000 rpm, at least about 8,000 rpm, at least about 7,000 rpm, at least about 6,000 rpm, at least about 5,000 rpm, at least about 4,000 rpm, at least about 3,000 rpm, at least about 2,000 rpm, at least about 1,000 rpm, at least about 900 rpm, at least about 800 rpm, at least about 500 rpm, at least about 600 rpm, at least about 500 rpm, at least about 400 rpm, at least about 300 rpm, at least about 200 rpm, at least about 100 rpm, at least about 75 rpm, at least about 50 rpm, at least about 25 rpm, at least about 15 rpm, or at least about 10 rpm.

In certain embodiments, the reaction of steps (iii) or (iv) further comprises heating above room temperature (e.g., above 25° C.). In certain embodiments, the reaction of steps (iii) or (iv) further comprises cooling (e.g., below 25° C.).

A suitable solution is a solvent or a solvent mixture that, in combination with the combined reacting partners and reagents, facilitates the interaction there-between. A suitable solution may solubilize or partially solubilize one or more of the reaction components, or, alternatively, the suitable solution may facilitate the suspension of one or more of the reaction components; see, generally, March (2001).

In certain embodiments, a suitable solution is an organic solvent. Exemplary organic solvents include hydrocarbons, halogen-containing hydrocarbons, polar protic solvents (e.g., alcohols), polar aprotic solvents (e.g., esters, ketones, aldehydes, sulfoxides, amides), nitriles, ethers, aromatic hydrocarbons, and mixtures thereof. Exemplary organic solvents include, but are not limited to, hexanes, pentanes, heptanes, cyclohexane, dichloromethane, chloroform, dichloroethane, polymeric alcohols (e.g., polyvinyl alcohol), methanol, ethanol, t-butylalcohol, isopropanol, ethyl acetate, isopropyl acetate, acetone, dimethyl sulfoxide (DMSO), dimethylformamide (DMF), dimethyl acetamide (DMA), acetonitrile, diethyl ether, methyl t-butyl ether (MTBE), tetrahydrofuran, dioxane, benzene, toluene, chlorobenzene, xylenes, and mixtures thereof. In certain embodiments, the organic solvent comprises dichloromethane. In certain embodiments, the organic solvent comprises dichloromethane and acetone. In certain embodiments, the organic solvent comprises acetone.

In certain embodiments, the suitable solvent of step (iv) is rapidly removed (e.g., within 1 minute) by evaporation. In certain embodiments, the suitable solvent is slowly removed (e.g., greater than 1 minute) by evaporation. In certain embodiments, the step of removing the solvent comprises removing by use of a vacuum (i.e., by rotary evaporation). In certain embodiments, the step of removing the solvent comprises removing by heating.

In other embodiments, the suitable solvent is evaporated by spray-drying. When the material is formed using spray-drying techniques, the resulting product may be a powder, a microparticle, a nanoparticle or a picoparticle.

The term “spray-drying” is used conventionally and broadly refers to processes involving breaking up liquid mixtures into small droplets (atomization) and rapidly removing the suitable solvent from the mixture in a spray-drying apparatus where there is a strong driving force for evaporation of solvent from the droplets. Spray-drying processes and spray-drying equipment are described generally in Perry's Chemical Engineers Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

The solvent-bearing feed can be spray-dried under a wide variety of conditions and yet still yield the inventive materials with desirable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of the common solvent) that they do not stick to or coat the spray-drying chamber wall. Examples of types of nozzles that may be used to form the solid amorphous dispersions include the two-fluid nozzle, the fountain-type nozzle, the flat fan-type nozzle, the pressure nozzle and the rotary atomizer.

The maximum droplet size varies widely as a function of the size, shape and flow pattern within the spray-dryer. In certain embodiments, droplets are less than about 500 pm in diameter upon exiting the nozzle.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas.

Generally, the energy for evaporation of solvent from the spray solution in a spray-drying process comes primarily from the drying gas. The drying gas can, in principle, be essentially any gas, but for safety reasons and to minimize undesirable decomposition of the material, an inert gas such as nitrogen, nitrogen-enriched air or argon is utilized. The drying gas is typically introduced into the drying chamber at a temperature between about 60° and about 300° C. or between about 80° and about 200° C.

Thus, in certain embodiments, the above step (iv) comprises:

(iv) stirring the mixture of one or more compounds and one or more biologically active agents or diagnostic agents with evaporation of the suitable solution by spray-drying to provide the material.

(iii) Tablets

In another embodiment, the inventive material is prepared as a tablet. Formation of tablets is well-known in the art; see, for example, Korhonen et al. AAPS PharmSciTech. (2002) 3:34; Picker-Freyer et al. AAPS PharmSciTech. (2006) 7:75; U.S. Pat. No. 4,880,585; and PCT application no. WO/1992/015204, each of which is incorporated herein by reference.

For example, one can mill, grind or press a compound of Formula I together with an additional agent (e.g., a biologically active agent or a diagnostic agent) and, optionally, a pharmaceutically acceptable excipient and/or solvent as described herein, to provide a tableted inventive material.

Thus, in certain aspects, the present invention provides a method of making a tableted inventive material, the method comprising:

(i) providing one or more compounds of the Formula I;

(ii) providing one or more biologically active agents or diagnostic agents, as described above and herein;

(iii) mixing one or more compounds and one or more biologically active agents or diagnostic agents to provide a mixture; and

(iv) tableting the mixture.

In certain embodiments, the compound of Formula I is in an amount greater than about 50 mg, about 60 mg, about 70 mg, about 80 mg, about 90 mg or about 100 mg, in the tableted material.

In certain embodiments, the tableting of step (iv) is by direct compression. In certain embodiments, the tableting of step (iv) is by direct compression using a single punch tablet press.

### Pharmaceutical Compositions

Once the complexes, micelles, liposomes, microparticles, nanoparticles, picoparticles, films, powders, etc. have been prepared, they may be combined with one or more pharmaceutical excipients to form a pharmaceutical composition that is suitable to administer to animals, including humans. In certain embodiments, the pharmaceutical composition is a personal care composition.

As would be appreciated by one of skill in this art, the excipients may be chosen based on the route of administration. Administration by be made by any known means, and includes, but is not limited to, transdermal administration, oral administration, parenteral administration, intravenus (IV) administration, intraarterial administration, by surgically implantion, by absorbtion, ingestion, injection, inhalation, or application to the skin, teeth, lips, eyes, etc.

Thus, in one aspect, the present invention provides a pharmaceutical composition comprising a material which comprises one or more compounds of Formula I; and a pharmaceutically acceptable excipient.

In one aspect, the present invention provides a personal care composition comprising a material which comprises one or more compounds of Formula I; and a pharmaceutically acceptable excipient.

In another aspect, the present invention provides a pharmaceutical composition comprising a material which comprises one or more compounds of Formula I and one or more biologically active agents; and a pharmaceutically acceptable excipient.

In another aspect, the present invention provides a personal care composition comprising a material which comprises one or more compounds of Formula I and one or more biologically active agents; and a pharmaceutically acceptable excipient.

In one aspect, the present invention provides a method of using an inventive material, the method comprising administering to a subject in need thereof a therapeutically effective amount of the inventive material.

In another aspect, the present invention provides a method of using an inventive material, the method comprising administering to a subject in need thereof a therapeutically effective amount of a pharmaceutical composition or personal care composition comprising the inventive material and a pharmaceutically acceptable excipient.

As used herein, a “subject in need thereof” refers to any animal suffering from, or having a susceptibility toward, a particular disease, disorder, or condition.

As defined herein, “therapeutically effective amount” refers to the amount or concentration of a biologically active agent present in a pharmaceutical composition or inventive material of the presently claimed invention, that, when administered to a subject, is effective to at least partially treat a condition from which the subject is suffering.

Exemplary diseases, disorders, or conditions which may be treatable by the inventive material or pharmaceutical compositions of the present invention include, but are not limited to, infectious diseases, external or internal lesions, sepsis, diseased tissue, bone or muscle injuries, bone breakage or fracture, joint conditions, arthritis, sepsis, necrosis, autoimmune diseases, blood disorders, bone disorders, cancers, circulation diseases, dental conditions, digestion and nutrition disorders, gastrointestinal diseases, genetic disorders, heart diseases, hormonal disorders, infectious diseases, inflammation, kidney diseases, liver diseases, mental health disorders, metabolic diseases, neurological disorders, skin disorders or conditions, pre-term labor, pain, headaches, and hormonal deficiencies or abnormalities.

Pharmaceutical compositions or personal care compositions/Formulations of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or Formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the Formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., microparticles, nanoparticles, liposomes, micelles, polynucleotide/lipid complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include beaty products, ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulations, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

### Methods of Use

Materials of the present invention may be useful (1) as a drug delivery devices (e.g., comprising a targeting biologically active agent) wherein the material may release a drug in a controlled manner without being compromised by a dynamic or static external environment; (2) as chewing gum or edible films for delivering biologically active agents; (3) as long-term circulating particles for applications including targeted drug delivery and blood substitutes; (4) as biodegradable particles, tubes, spheres, strands, threads, coiled strands, films, sheets, fibers, meshes, and the like, (5) as sutures which are tunable for fast or slow degradation and/or surgical glue and adhesives; (6) as a medical implant coatings (e.g., stents); (7) as an injectable; (8) for microfabrication applications (capillary networks, diagnostics) and/or for tissue engineering (i.e., bladder, bone, brain, skin, cardiac tissue, ligament, cartilage, tendon, genital, muscle, artery, veins, kidney, pancreas, liver, intestine, stomach, and other tissues); (9) for topical applications; (10) as a component in aerosols; (11) as a cosmetics and/or personal use item; (12) as a material in food Formulations; (13) as a prophylactic; (14) as a diagnostic agent; and (15) as an aid for agriculture or landscaping (e.g., seeding or releasing seeds, releasing insecticides, and the like); (16) controlled release of small molecules for local or systemic needs; (17) controlled release of macromolecules (proteins, nucleic acids) for local or systemic needs; (18) the controlled release of vaccines, and the controlled release of peptides. The present invention contemplates, but is not limited to, all such useful applications for materials of the present invention, and pharmaceutical compositions comprising such inventive materials.

Specific anticipated uses of the materials include, but are not limited to, the controlled local release of steroids to treat nasal polyps, the controlled local release of immunomodulators to treat dry eye in canines, and the controlled release of agents to treat ocular diseases.

### EXAMPLES

These and other aspects of the present invention will be further appreciated upon consideration of the following Examples, which are intended to illustrate certain particular embodiments of the invention but are not intended to limit its scope, as defined by the claims.

### Example 1

### Synthesis of Compounds and Construction of Materials

1,3-Dihydroxyacetone dimer (DHA), hexadecanoyl chloride, tetradecanoyl chloride, dodecanoyl chloride, decanoyl chloride, ocatanoyl chloride, chloroform, rhodamine-B, anhydrous pyridine and nile red were all obtained from Sigma-Aldrich (Saint Louis, Mo.) and used without further purification. Poly(vinyl alcohol) (PVA, MW˜25000, 88 mole % hydrolyzed) was purchased from Polysciences Inc. Dichloromethane (DCM), acetone, tetrahydrafuran (THF) and diethyl ether were purchased from J. T. Baker (West Chester, Pa.).

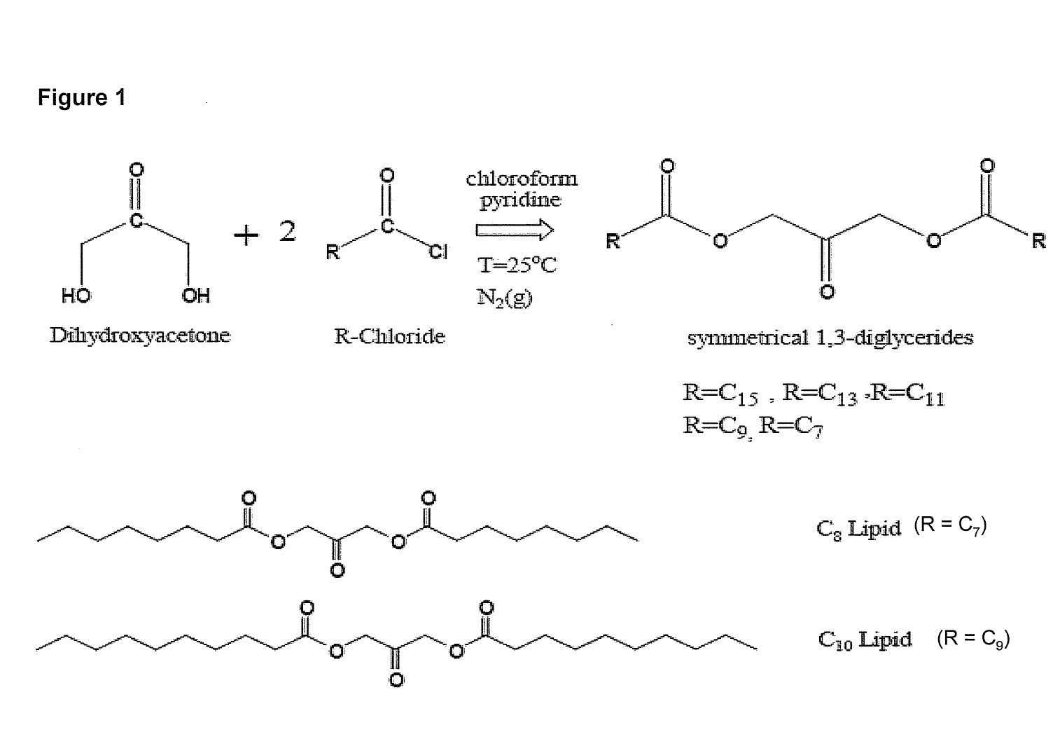

Synthesis of symmetrical 1,3-diglycerides. Symmetrical 1,3-diglycerides were synthesized through modification of a previously reported method by Bentley and McCrae (23). The general procedure is as follows, using the synthesis of C₁₆ symmetrical diglyceride as an example: DHA (3.5 gm, 39 mmole) was stirred in 150 ml of chloroform under flow of N₂ at room temperature, followed by sequential dropwise addition of hexadecanoyl chloride (22.1 ml, 80 mmol) and anhydrous pyridine (7.5 ml), in that order. The mixture was stirred for 3 hrs at room temperature followed by extraction (2×) of the chloroform layer with 150 ml water portions. Chloroform was evaporated by rotary evaporation and the remaining solid was re-crystallized using methylene chloride-diethyl ether (1:1), FIG. 1. ¹H-NMR spectra were recorded at room temperature with a Brucker AF-300 spectrometer operating at 300.13 MHz.

Fabrication of solid lipid microparticles (SLM) by the spontaneous emulsification-solvent evaporation method. Symmetrical 1,3-diglyceride (0.1 gm) was dissolved in a 3:2 (v:v) ratio of DCM:acetone by vigorous vortex for approximately 10 seconds. The lipid solution was transferred using a glass pipet into 450 ml of 2.5% PVA solution stirring at 800 rpm and stirred for 3 hrs to allow full evaporation of all organic solvents. Particles were separated by centrifugation at 4800 RCF for 60 minutes followed by multiple washes (3×) with deionized water and re-centrifugation at 4800 RCF for 45 minutes. The particles were resuspended in small volumes of deionized water and lyophilized for a minimum of 12 hrs and were stored at −20° C. in the presence of desiccant. The final products were fine powder particles with approximate yield of 75% (relative to original weight of lipid).

Particle characterization. Particle morphology and size were studied at low voltage (5 kV) using scanning electron microscopy imaging (LEICA 440) after coating with palladium. Surface charge measurements on samples suspended in 1:10 diluted PBS were carried out using a Malvern Zetasizer-Nano ZS (Malvern, UK). A PRECO Hydraulic press (Los Angles, Calif.) was used to make lipid pellets with approximate surface area of 25 mm². For contact angle measurements, a ramé-hart contact angle goniometer (100-FO) was used to measure the sessile contact angle of water on a lipid pellet surface. Unless otherwise stated, measurements were taken in triplicate.

Encapsulation efficiency of SLM. The encapsulation efficiency of lipid microparticles was determined by complete drug recovery from melted microparticles, followed by comparison with the theoretical maximum drug loading. For microparticles containing rhodamine-B, encapsulated drug was recovered from 5.0 mg of lipid particles by melting in 1-ml of phosphate buffered saline (PBS) at five degrees above the corresponding lipid melting point. The resulting emulsion was cooled to room temperature and any re-solidified particulate matter was removed by centrifugal filtration (Ultrafree-MC, Millipore). The concentration of rhodamine-B in the supernatant was quantified in a microplate spectrofluorometer (Spectramax GeminiEM; Molecular Devices, Sunnyvale, Calif.) using 96-well black assay plates (Corning, Inc). The excitation and emission wavelengths were 540 nm and 625 nm, respectively. Complete recovery of nile-red from particles was performed in a similar fashion, with the exception that particles were melted in ethanol due to the insolubility of nile red in PBS. The excitation and emission wavelengths of nile red were 550 nm and 650 nm, respectively.

In vitro drug release and model drug distribution. In vitro drug release was determined by suspending 5.0 mg of microspheres in 1-ml of PBS in amber microcentrifuge tubes (Eppendorf) and incubating at 37° C. with rotation (60 rpm). For rhodamine-B, samples were filtered, and the fluorescence was quantified in a microplate spectrofluorometer at each given time interval. Rhodamine-B loaded samples were discarded after each time interval reading, thus each data point represents three dedicated samples. In the case of nile red, samples were first centrifuged (16,000 RCF) to pellet solids. The supernatants were then removed for fluorescence measurements, and pellets were resuspended in fresh buffer and allowed to rotate until the next time interval.

Distribution profiles of rhodamine-B and nile red within lipid microparticles were obtained by laser confocal microscopy (Leica TCS SP2) with a helium-neon laser excitation source (543 nm). Samples were placed on microscope slides and sealed with cover slips. Fluorescence intensity profiles were determined with image analysis software (Image J).

Statistical Analysis. The statistical significance of experimental results was determined using the two sample Students' t-test with p<0.05. The calculated errors were set to standard mean error for all experimental results.

Lipid characterization. Symmetrical 1,3-diglycerides were successfully synthesized following the Bentley and McCrae synthetic route (Bentley and McCrae, J. Org. Chem. 35 (1970) 2082-2083) with yields in excess of 70% (Scheme 1). Acyl chlorides were chosen such that the final product was derived from physiological lipids; for example palmitoyl chloride (IUPAC: hexadecanoyl chloride), derived from hexadecanoic acid, a fatty acid freely present in various metabolic pathways, was selected to synthesize C₁₆ lipid. In addition, all of the synthesized lipids have melting points that are above room temperature, making them good candidates for fabrication into powders or particulates for drug delivery.

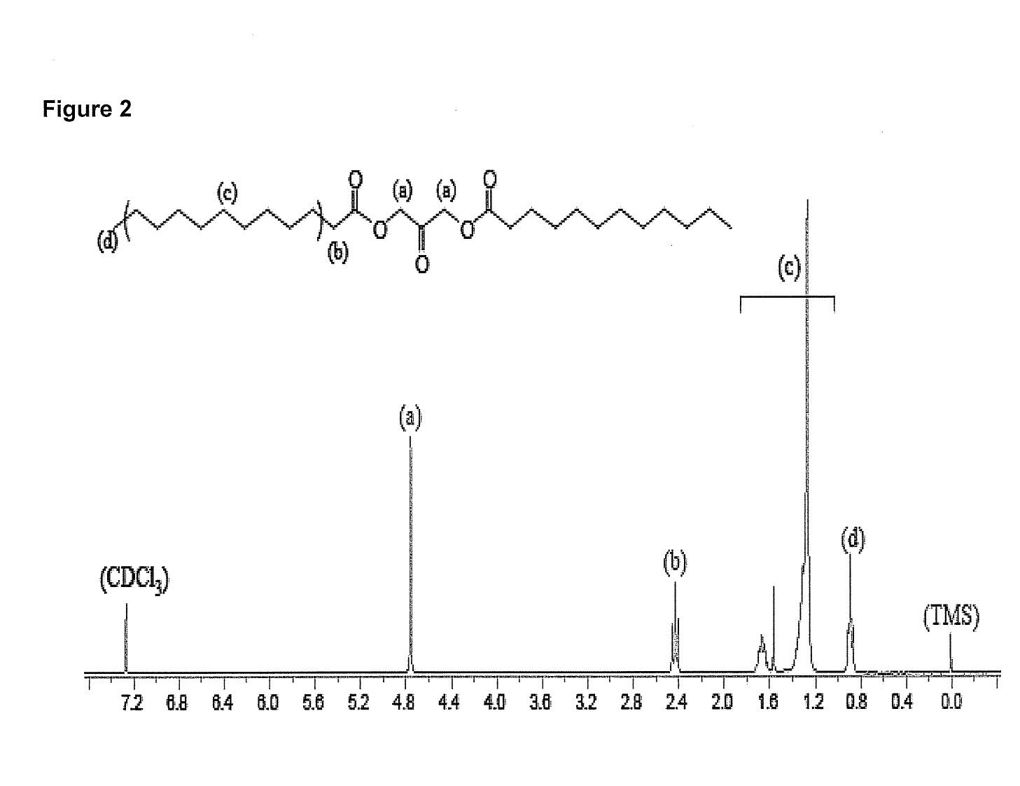

Elemental analysis for C₈-C₁₆ lipids were all within acceptable ranges (Table 1). Proton nuclear magnetic resonance (¹H NMR) was also utilized to further characterize the products. Chemical shifts (δ) all correlated to the proposed structures (FIG. 2).

TABLE 1

| | | Melting | E. analysis - | | E. analysis - | |
|---|---|---|---|---|---|---|
| Sample | | Point | theoretical | | experimental | |
| Name | Lipid | (° C.) | % C | % H | % C | % H |
| C₁₆ | C₃₅H₆₆O₅ | 76-78 | 74.13 | 11.756 | 74.05 | 11.41 |
| C₁₄ | C₃₁H₅₈O₅ | 73-76 | 72.87 | 11.46 | 73.18 | 11.54 |
| C₁₂ | C₂₇H₅₀O₅ | 69-70 | 71.32 | 11.08 | 71.32 | 11.14 |
| C₁₀ | C₂₃H₄₂O₅ | 64-65 | 69.31 | 10.62 | 69.30 | 10.81 |
| C₈ | C₁₉H₃₄O₅ | 56-58 | 66.63 | 10.01 | 66.57 | 10.23 |

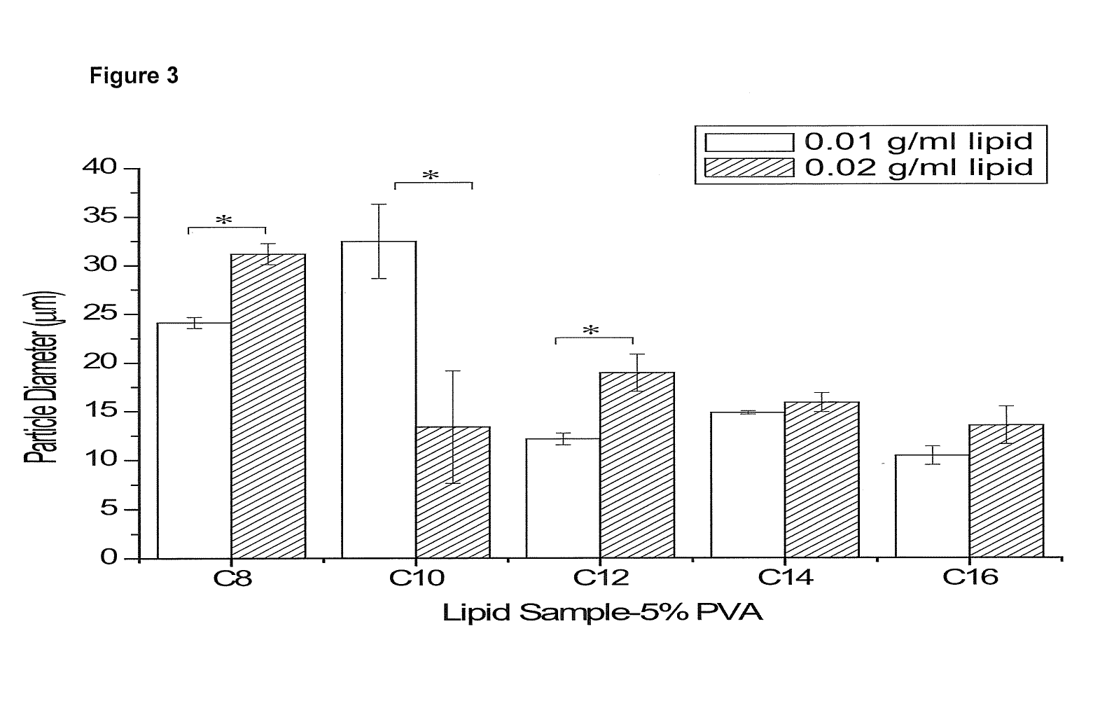

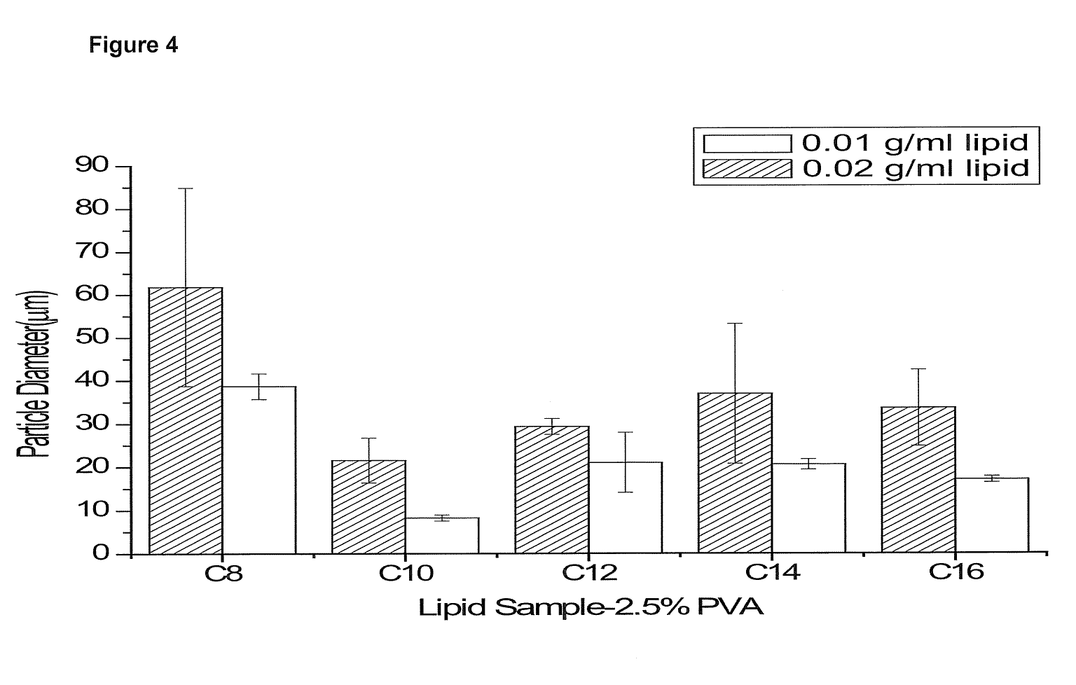

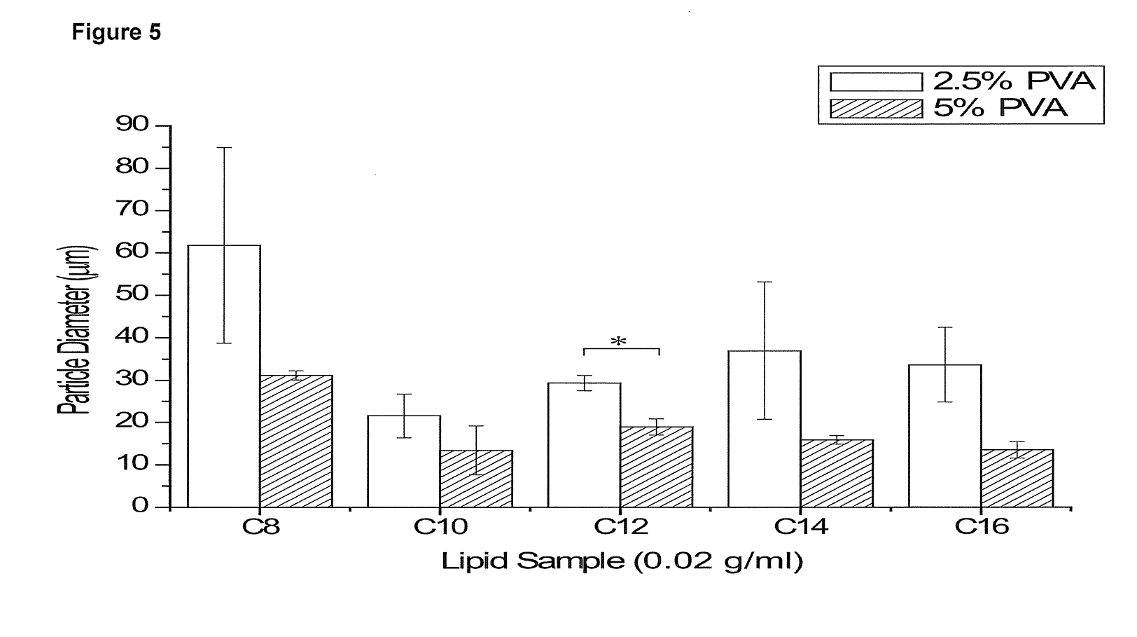

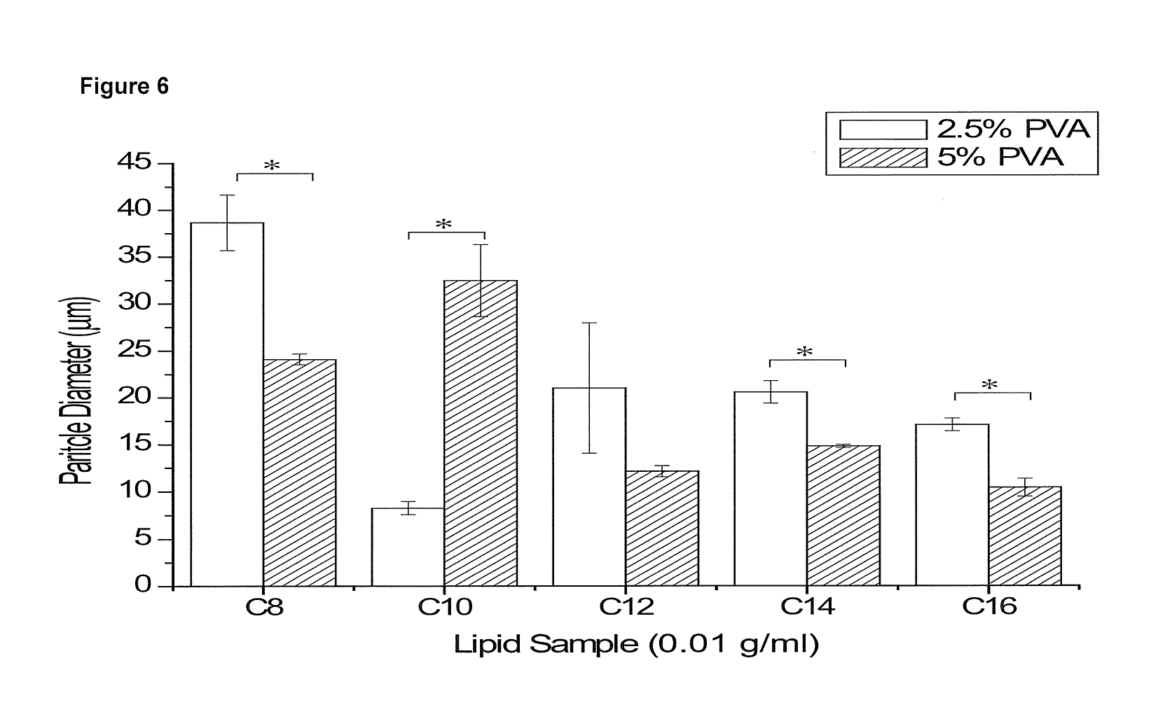

SLM size characterization. Particle sizes were measured using scanning electron microscopy (SEM) images that were obtained at low voltage (5 kV). Sizing results collected from multiple image analyses using Image J software showed polydisperse particle distributions in the micron size range. The effect of lipid concentration and surfactant concentration on the final particle size with constant stirring rate and temperature was determined by serial variation of both parameters. The results showed that increasing the lipid concentration led to an increase in mean diameter (FIGS. 3 and 4), whereas increasing the surfactant concentration did not significantly change particle size (FIGS. 5 and 6). Statistical significance of particle size as a function of lipid chain length was inconclusive because of the polydispersity of the particle size distribution.

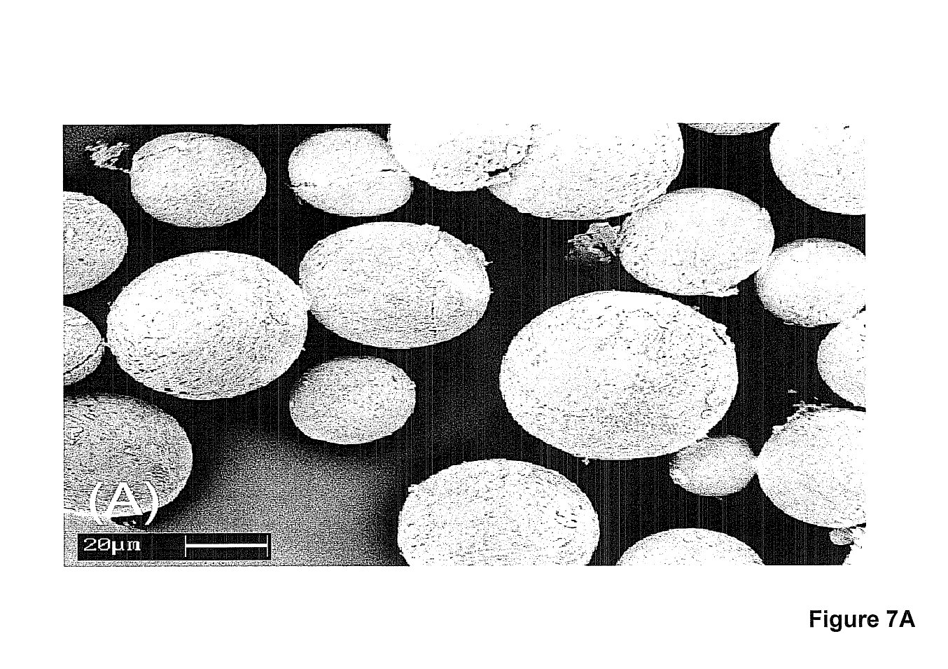

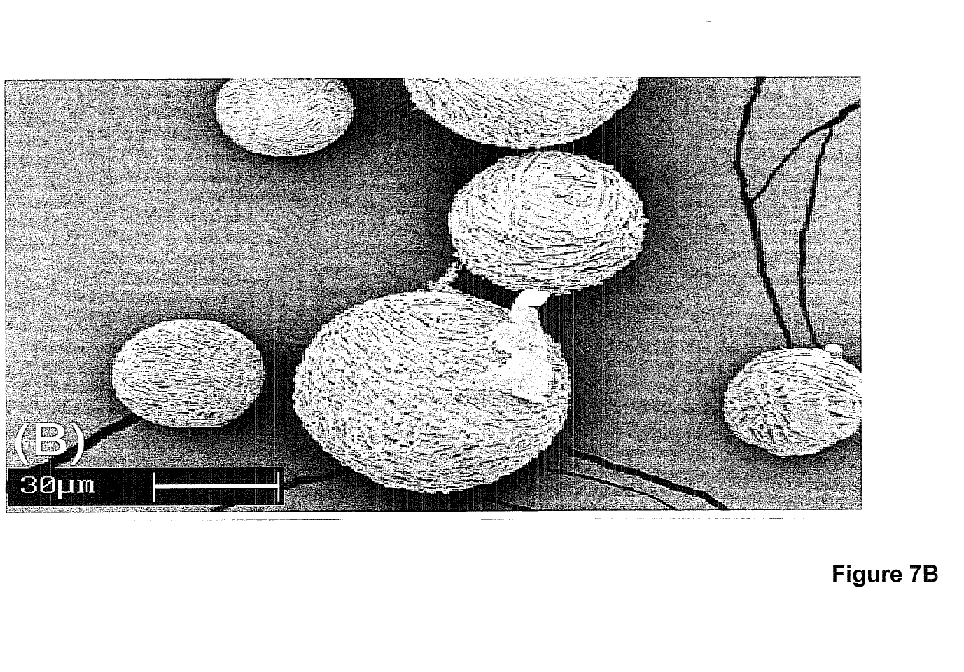

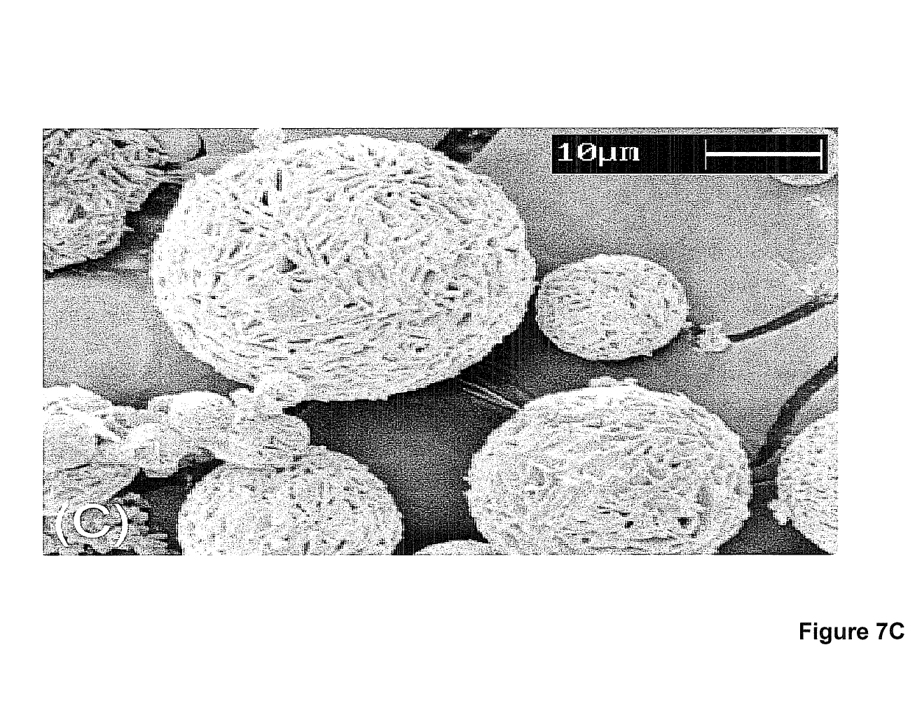

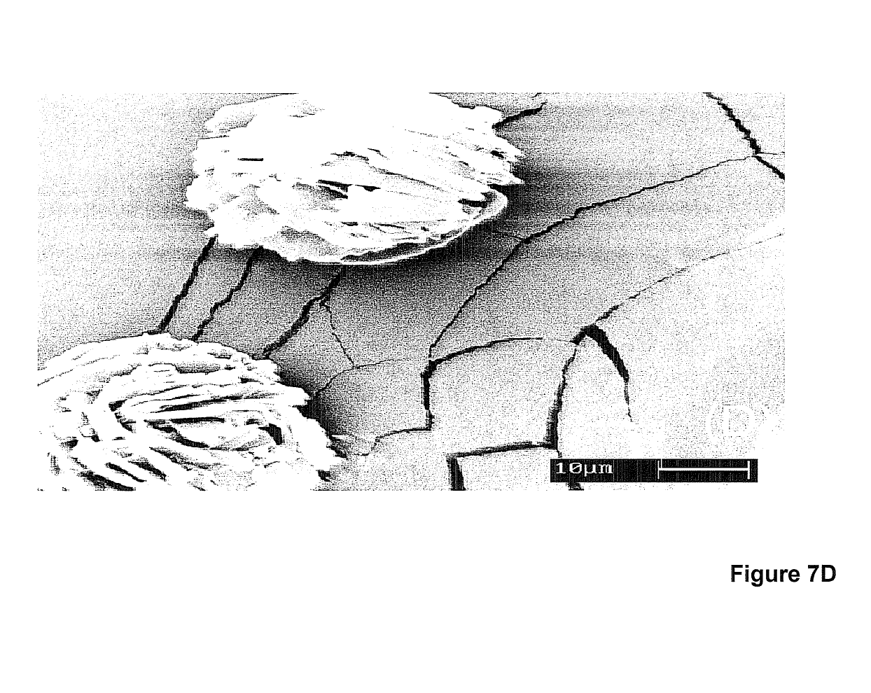

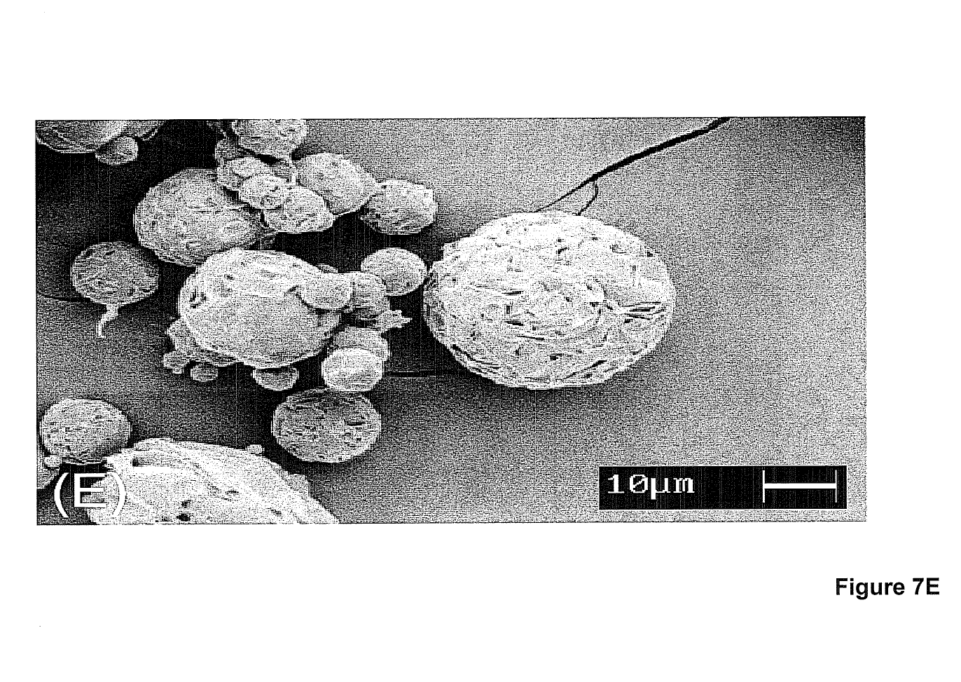

SLM Morphology and Surface Characterization. The particle surfaces were irregular with unique patterns depending on the lipid chain length. In spontaneous emulsification, the solvent must diffuse out into the external aqueous phase prior to evaporation. The irregular surface morphology of the lipid particles may therefore be a result of rapid solvent removal, leading to local precipitation or crystallization of the lipids (Jain, Materials 21 (2000) 2475-2490). Further inspection of the particle surfaces revealed that the morphology is distinct for each lipid chain length. The smaller lipids (C₈ and C₁₀) show a relatively smooth surface compared to C₁₂ through C₁₆. Additionally, the surface structures become more prominent as the lipid chin length increases (FIG. 7).

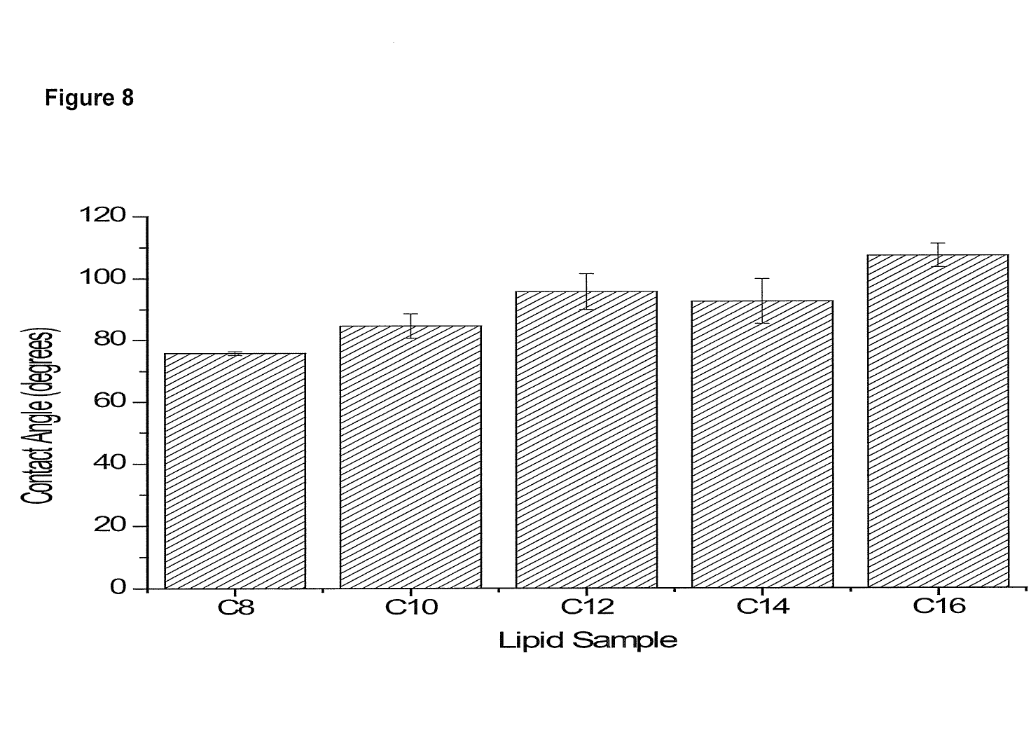

Because hydrophobicity of the lipid matrix can influence encapsulation efficiency and release kinetics, we measured the sessile surface contact angle of water on pellets fabricated from each lipid. All surfaces were hydrophobic as expected, with hydrophobicity increasing with increasing lipid chain length (FIG. 8).

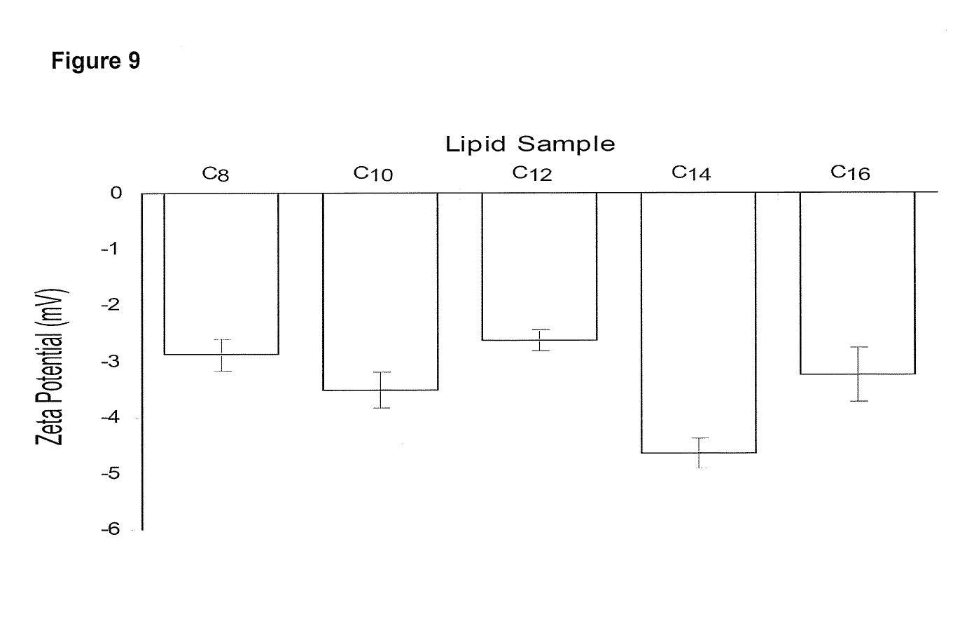

SLM Surface charge. Zeta potential measurements showed that the SLMs are negatively charged in a 1:10 dilution of PBS in water. The results indicate the presence of hydrolyzed ester groups on the surface. Along with hydroxyl PVA side chains, the ester groups of the diglycerides likely rearrange in such manner to associate with the aqueous phase. Upon removal of the organic solvent, these ester groups remain exposed on the surface of the newly formed lipid particles, leading to their hydrolysis and exposure of negatively charged carboxyl groups (FIG. 9).

SLM Encapsulation Efficiency. Rhodamine-B (a hydrophilic model drug) and nile red (a hydrophobic model drug) were successfully encapsulated in lipid particles, although with very different efficiencies. For rhodamine-B loaded particles, the encapsulation efficiency was less than 10%, whereas for nile red the efficiency exceeds 70% (FIGS. 10 and 11). Factors that impact encapsulation efficiency in microparticles range from the method of preparation to the nature of the interaction between the model drug and the encapsulating matrix. Low encapsulation efficiency in the case of rhodamine-B can be attributed to the hydrophobicity of the lipid matrix, the porous nature of the lipid microparticles, as well as the solubility of rhodamine-B in the aqueous PVA solution. Amongst all lipids, C₈ with the shortest lipid chain displayed the highest encapsulation efficiency of rhodamine-B. Based on contact angle measurements that show that C₈ lipid is the least hydrophobic of the lipids, and the SEM images that display the relatively smooth morphology of C₈-derived particles, it is expected that the C₈ particles should entrap rhodamine-B to a greater extent than the longer and more hydrophobic lipid counterparts (FIGS. 7 and 8).

For nile red the results are very different. All lipids encapsulated significant amounts of nile red (>70%). Additionally the Students' t-test analysis of encapsulation efficiency for nile red loaded lipid particles showed that the difference between the lipids is not statistically significant (p>0.05). The high encapsulation efficiency in the case of nile red is attributed to a more favorable interaction between the hydrophobic model drug and the encapsulating lipid matrix.

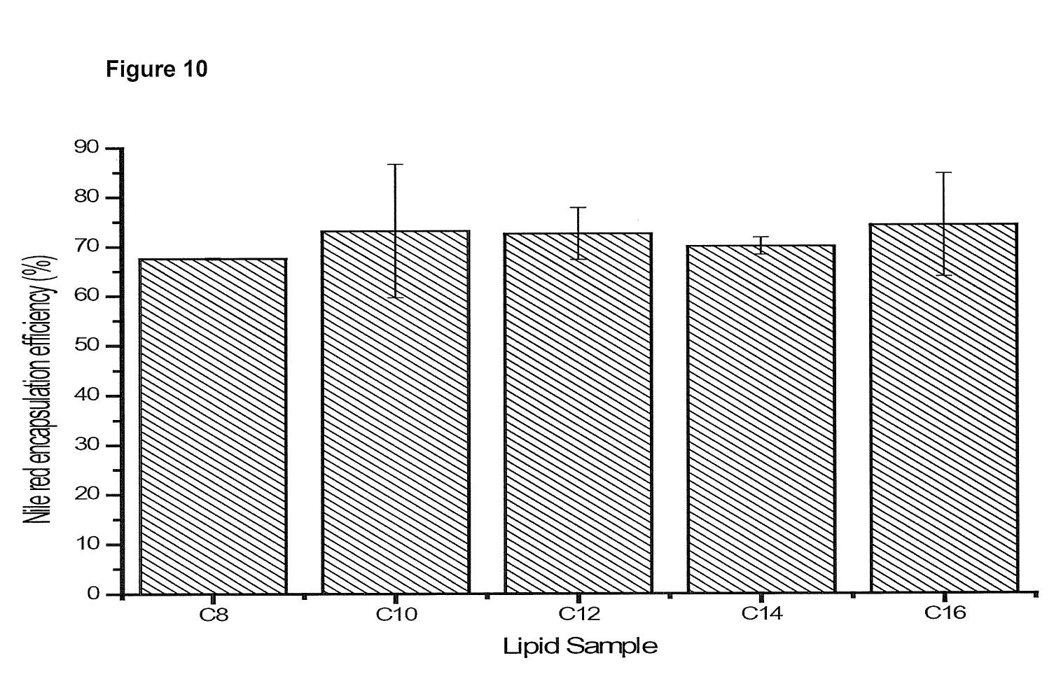

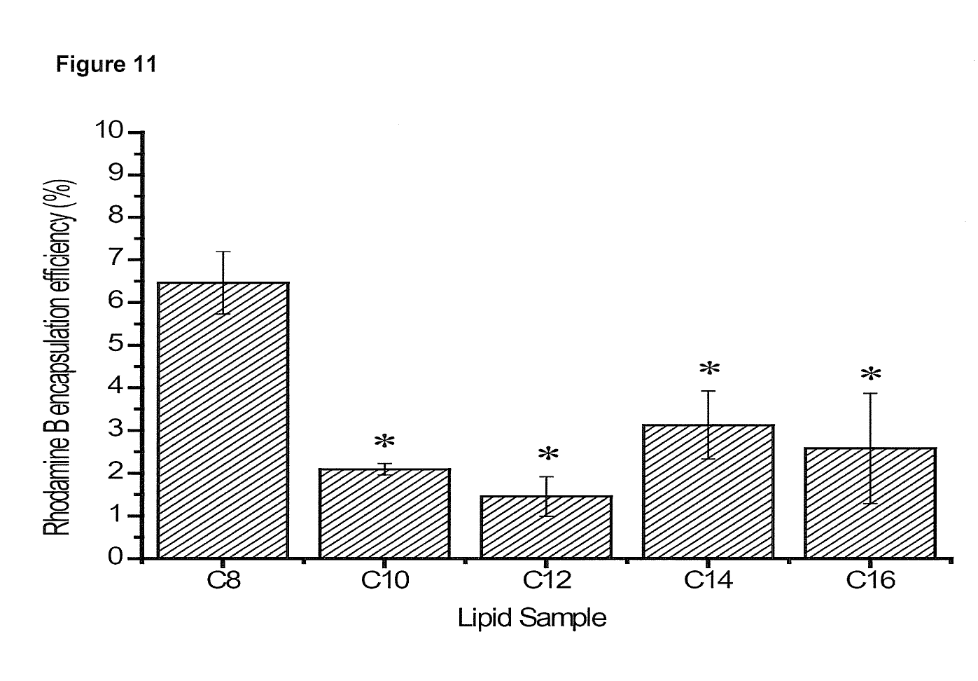

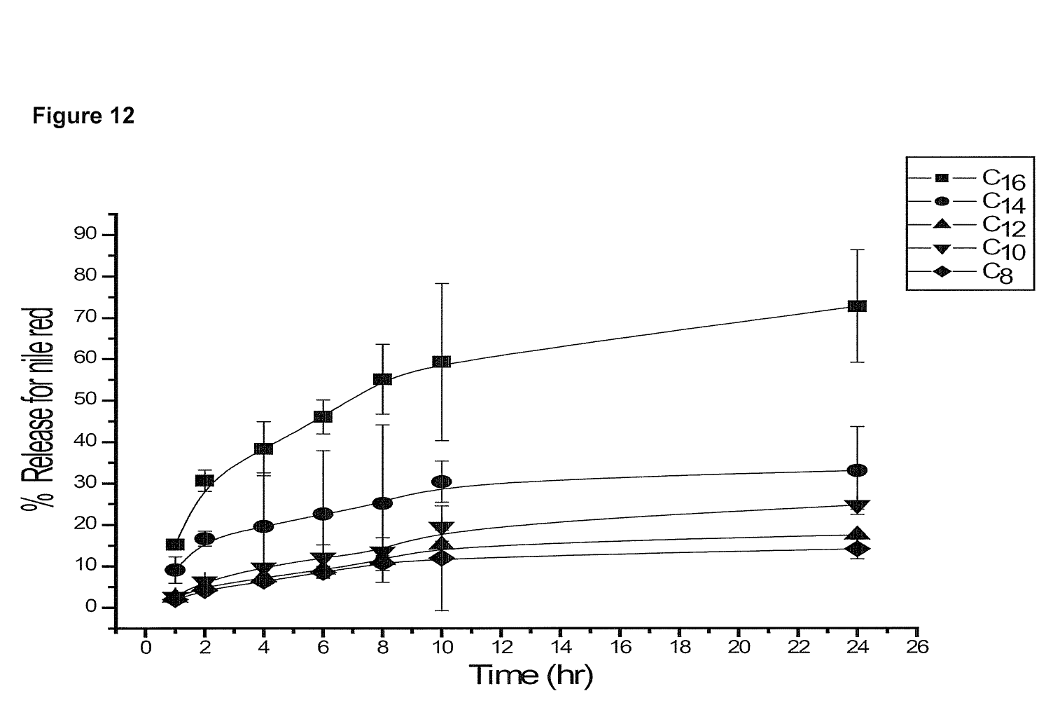

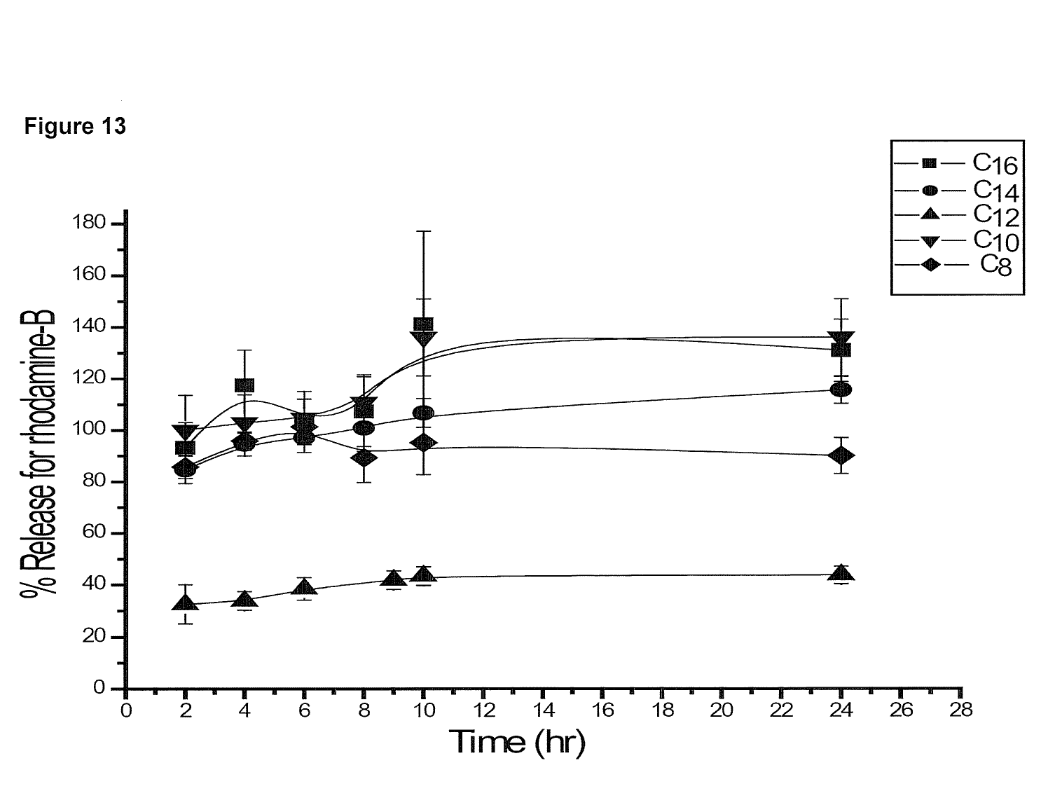

In vitro controlled release behavior. The in vitro release characteristics of lipid microparticles encapsulating nile red and rhodamine-B at 37° C. were observed over a 24 hour period (FIGS. 12 and 13). In general, the release rates of nile red increased with increasing lipid chain length, while rhodamine-B exhibited burst release in all cases.

Particles loaded with the hydrophobic model compound nile red exhibited lipid chain length-dependent release behavior, with longer chain lipid particles favoring faster release kinetics (FIG. 12). This release behavior appears to be related to microparticle surface morphology, which apparently overrides the influence of lipid hydrophobicity. Slower release in the case of shorter lipid chain lengths (i.e. C₈-C₁₂) may be a result of their densely formed microstructures and smooth morphology, thereby limiting solvent penetration into the particles. The increased porosity in longer chain lipid microparticles (i.e. C₁₄-C₁₆) facilitates faster release kinetics through the increase in available surface area, despite the expected tendency for more hydrophobic lipids to retain the hydrophobic model drug.

Particle size is a key determinant of drug release and matrix degradation (Berkland et al., Pharm. Res. 20 (2003) 1055-1062). Larger particles have smaller surface:volume ratios, leading to an expectation that larger particles should exhibit decreased release rates. Particles prepared using the solvent emulsification evaporation method, however, were fairly polydisperse, making it difficult to further mechanistically evaluate the release profiles. Additionally, during the fabrication process, smaller particles also tend to harden faster, potentially impacting the drug distribution within the lipid matrix and consequently impacting the release behavior.

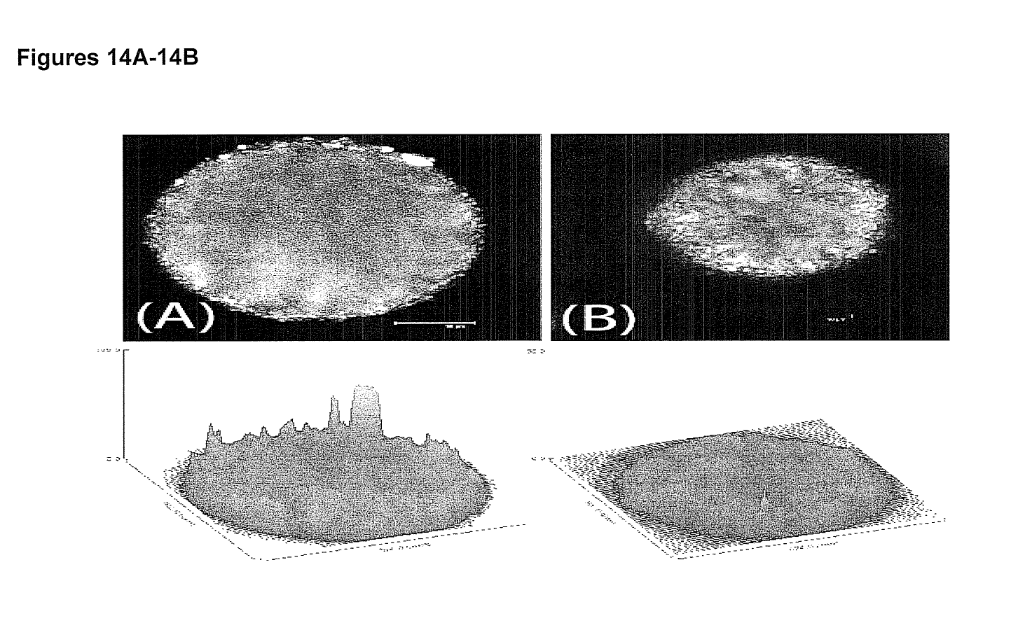

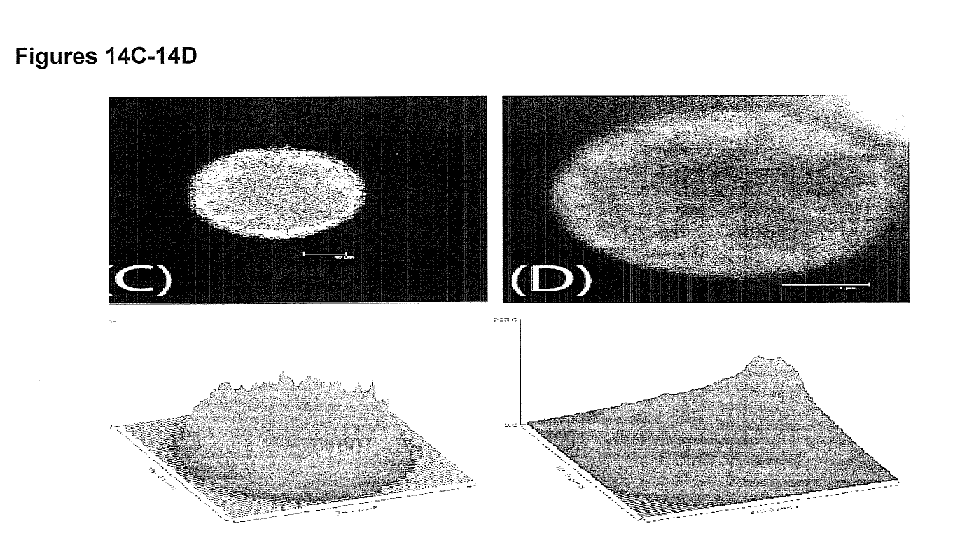

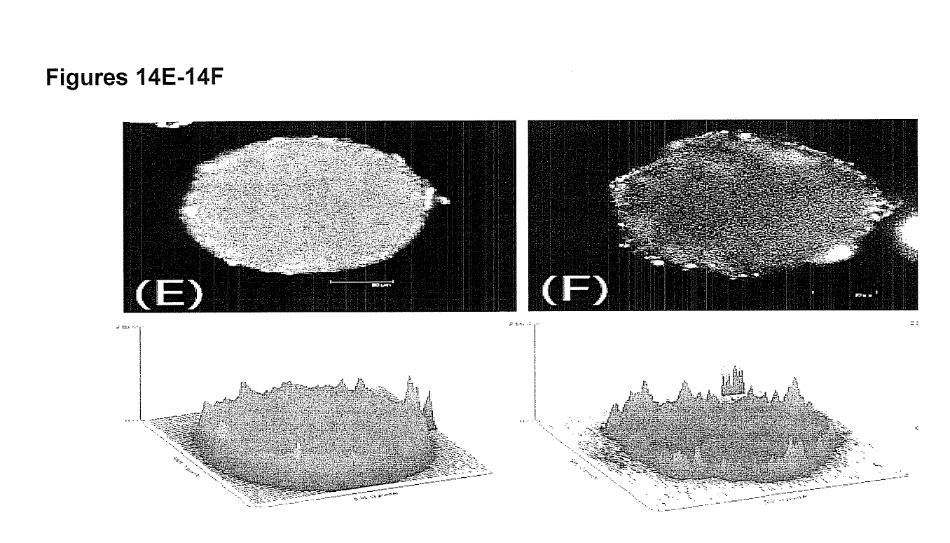

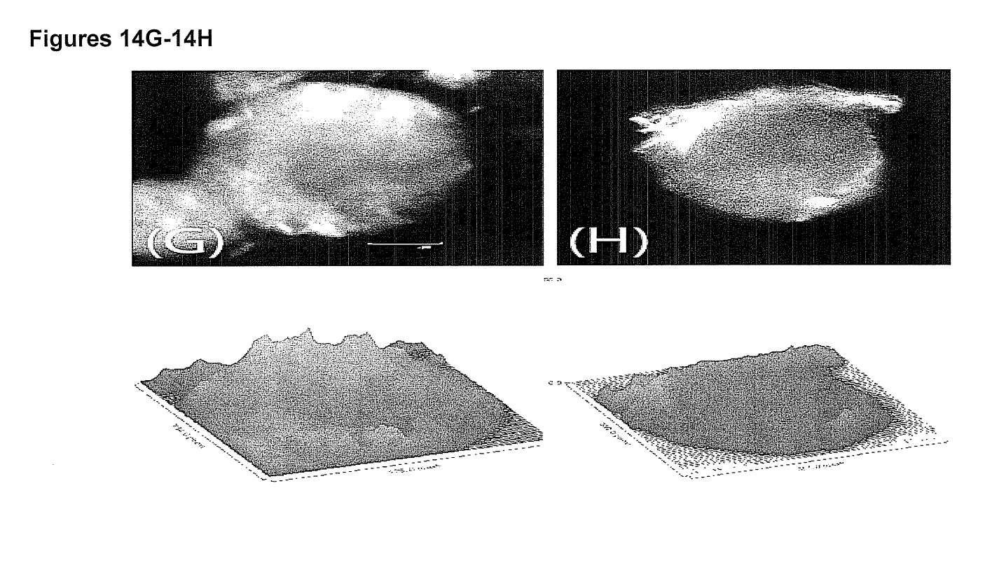

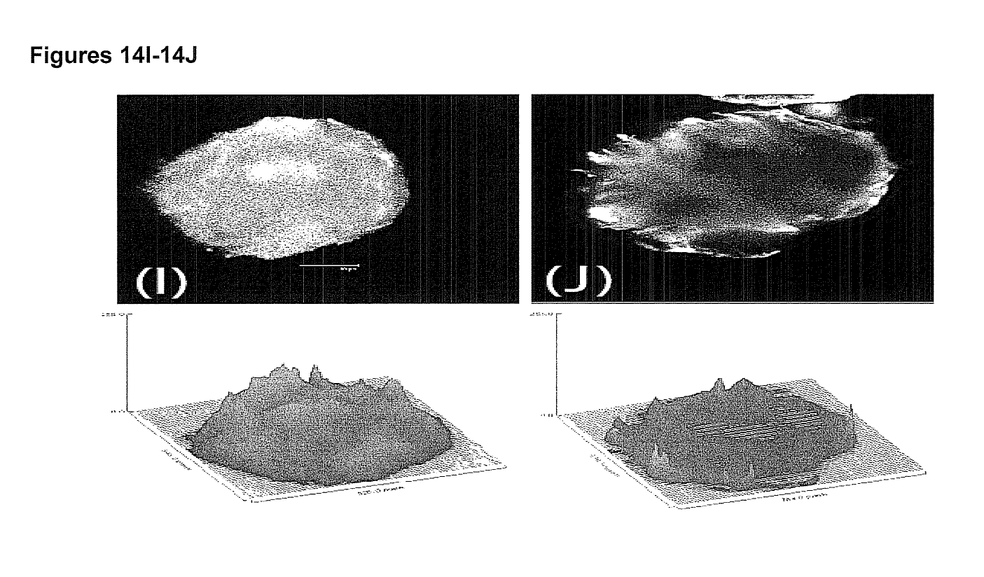

To ascertain whether the intra-particle drug distribution may impact the release behavior of nile red in lipid particles, images were taken by confocal laser scanning microscopy (FIG. 14). Nile red-loaded particles revealed that for longer lipid chain lengths, the hydrophobic model drug is homogeneously distributed throughout the lipid matrix, with occasional patches of concentrated drug localized on the particle surface. The presence of this surface-localized nile red may contribute to the larger initial release rates in longer-chain lipid particles. In particles from shorter chain length lipids, nile red was still homogenously distributed throughout the particle, however any surface concentration of drug was decidedly absent.

The release of the hydrophilic model compound rhodamine-B from microparticles exhibited burst release behavior at all lipid chain lengths, with the rapid expulsion of the encapsulated drug in the initial few hours of the study (FIG. 13), with the exception of C₁₂-derived particles which displayed much slower release in comparison to other lipid particles. The underlying explanation behind the observed idiosyncratic release behavior in the case of C₁₂-derived microparticles is unclear. Confocal images of the particles containing rhodamine-B showed that this hydrophilic model drug is primarily distributed on the particle surface, likely minimizing interaction with the hydrophobic lipid matrix. The burst release behavior of these particles suggests that the surface-localized rhodamine-B is rapidly released into the surrounding aqueous medium (FIG. 14). In future work, we plan to establish methods by which to gain greater control over the particle size and surface porosity, thereby providing level ground work for comparison amongst different lipid chain length particles and the impact of porosity on in vitro controlled release behavior.

### Example 2

### Fabrication of Tablets

Tablets consisting of the symmetrical lipids were manufactured by direct compression using a Stokes single punch tablet press. One hundred milligrams of each lipid was charged into the tablet press and compressed to form a solid tablet. A model drug compound, tartrazine, was homogeneously entrapped within the tablet to mimic a therapeutic drug.

### Example 3

### Microparticle Formulation with a Biologically Active Agent

Lipid microparticles containing dexamethasone were formulated by the method of spontaneous emulsification. In the present example, the symmetrical lipid comprised of dihydroxyacetone and lauric acid (HOC(═O)C₁₀CH₃) was used.

Lipid comprised of dihydroxyacetone and lauric acid:

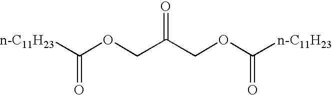

The lipid (0.1 g) and dexamethasone (0.01 g) was dissolved in a 3:2 (v:v) ratio of dichloromethane:acetone by vigorous vortex for approximately 10 seconds. The solution was transferred dropwise using a glass pipet into a stirring solution of polyvinylacohol (450 ml, 2.5%). The suspension was stirred for 3 hrs to allow evaporation of the organic solvents. Particles were isolated by centrifugation at 4,800 RCF for 60 minutes followed by multiple washes (3 times) with deionized water and re-centrifugation at 4800 RCF for 45 minutes. The particles were resuspended in 10 mL of deionized water and lyophilized for a minimum of 12 hrs.

These particles were designed to be administered by injection or surgical implantation. A specific application of the formulation is for the treatment of nasal polyps. The microparticles are to be administered in the nasal cavity in close proximity to one or more polyps and are designed to locally release dexamethasone over time to treat and prevent inflammation and polyps growth.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A material comprising a biologically active agent or a diagnostic agent, and one or more compounds of the Formula I: 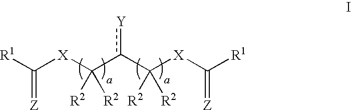
wherein: [symbol]
Y is, independently, ═O, —OR^{O}, ═S, —SR^{S}, ═NR^{N}, or —N(R^{N})₂, wherein R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group; R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thiol protecting group; and each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;
each instance of X is, independently, —O—, —S—, or —N(R^{N})—;
each instance of Z is, independently, O, S, or N(R^{N});
each instance of R¹ is, independently, cyclic or acyclic, substituted or unsubstituted aliphatic; cyclic or acyclic, substituted or unsubstituted heteroaliphatic; substituted or unsubstituted aryl; or substituted or unsubstituted heteroaryl;
each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and
each instance of a is, independently, an integer between 1 to 6, inclusive.

2. The material according to claim 1, wherein compound is of the Formula I-1: 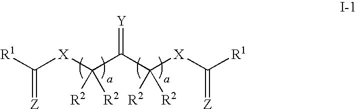
wherein [symbol]
Y is ═O, ═S, or ═NR^{N}, wherein R^{N} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group.

3. The material according to claim 2, wherein said compound is of the Formula I-3:

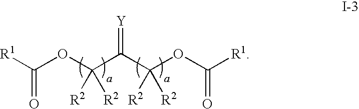

4. The material according to claim 3, wherein said compound is of the Formula I-6:

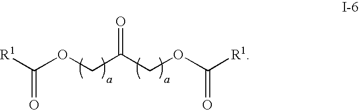

5. The material according to claim 1, wherein a is 1.

6. The material according to claim 1, wherein each R¹ group is the same.

7. The material according to claim 1, wherein each R¹ group is different.

8. The material according to claim 1, wherein each instance of R¹ is, independently, a cyclic or acyclic, substituted or unsubstituted C₁₋₃₂ aliphatic.

9. The material according to claim 1, wherein each instance of R¹ is, independently, a cyclic or acyclic, substituted or unsubstituted C₁₋₃₂ heteroaliphatic.

10. The material according to claim 1, wherein each instance of R¹ is independently, a cyclic or acyclic, substituted or unsubstituted C₁₋₃₂ alkynyl.

11. The material according to claim 1, wherein each instance of R¹ is independently, a cyclic or acyclic, substituted or unsubstituted C₁₋₃₂ alkenyl.

12. The material according to claim 1, wherein each instance of R¹ is, independently, a cyclic or acyclic, a substituted or unsubstituted C₁₋₃₂ alkyl.

13. The material according claim 12, wherein each instance of R¹ is, independently, an even-numbered C₂₋₃₂ alkyl group.

14. The material according to claim 12, wherein each instance of R¹ is, independently, an odd-numbered C₁₋₂₅ alkyl group.

15. The material according to claim 1, wherein the compound is:

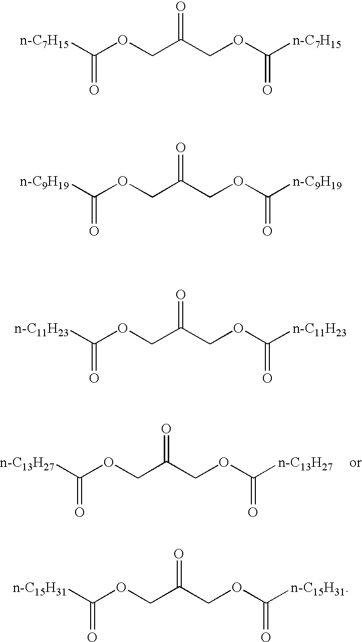

16. The material according to claim 1, wherein said biologically active agent is a small organic molecule, a therapeutic cell, an amino acid, a dipeptide, a peptide, a polypeptide, a protein, an enzyme, a carbohydrate, a monosaccharide, an oligosaccharide, a polysaccharide, a nucleoprotein, a mucoprotein, a lipoprotein, a small molecule linked to a protein, a glycoprotein, a steroid, a nucleic acid, a DNA, an RNA, a nucleotide, a nucleoside, an oligonucleotide, an antisense oligonucleotide, a lipid, a hormone, or a vitamin.

17. The material according to claim 16, wherein said small organic molecule is a small organic hydrophobic molecule.

18. The material according to claim 16, wherein said small organic molecule has a molecular weight of less than about 1500 g/mol.

19. The material according to claim 1, wherein the material is a tablet, a particle, a film, a sheet, a coating, a micelle, a liposome, a rod, a tube, a spheroid, a cone, a composite or a matrix.

20. The material according to claim 19, wherein the particle is a microparticle or a nanoparticle.

21. The material according to claim 19, wherein the material is a tablet.

22. The material according to claim 20, wherein the microparticle is a solid lipid microparticle.

23. A pharmaceutical composition comprising a material of claim 1 and a pharmaceutically acceptable excipient.

24. A method of using a pharmaceutical composition of claim 23, said method comprising administering to a subject in need thereof a therapeutically effective amount of said pharmaceutical composition.

25. A method of using a material of claim 1, said method comprising administering to a subject in need thereof a therapeutically effective amount of said material.

26. A method of making a material of claim 1, said method comprising:
providing one or more compounds having the Formula I: 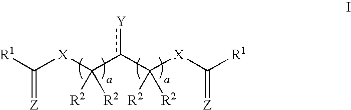
wherein: [symbol]
Y is, independently, ═O, —OR^{O}, ═S, —SR^{S}, ═NR^{N}, or —N(R^{N})₂, wherein R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group; R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thiol protecting group; and each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;
each instance of X is, independently, —O—, —S—, or —N(R^{N})—;
each instance of Z is, independently, O, S, or N(R^{N});
each instance of R¹ is, independently, cyclic or acyclic, substituted or unsubstituted aliphatic; cyclic or acyclic, substituted or unsubstituted heteroaliphatic; substituted or unsubstituted aryl; or substituted or unsubstituted heteroaryl;
each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and
each instance of a is, independently, an integer between 1 to 6, inclusive;
providing a biologically active agent or a diagnostic agent;
adding said one or more compounds and said biologically active agent or diagnostic agent to a suitable solution to provide a mixture; and
stirring said mixture with evaporation of said suitable solution.

27. The method of claim 26, wherein said stirring step comprises a stirring at speeds of at least about 50 revolutions per minute (rpm).

28. The method of claim 26, wherein the evaporation of step (iv) is by spray-drying.

29. A method of making a tableted inventive material, the method comprising:
providing one or more compounds of the Formula I: 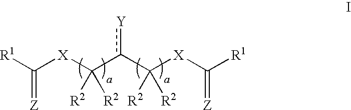
wherein: [symbol]
Y is, independently, ═O, —OR^{O}, ═S, —SR^{S}, ═NR^{N}, or —N(R^{N})₂, wherein R^{O} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable hydroxyl protecting group; R^{S} is hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; or a suitable thiol protecting group; and each instance of R^{N} is, independently, hydrogen; cyclic or acyclic, substituted or unsubstituted aliphatic; cyclic or acyclic, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted amino; or a suitable amino protecting group; or two R^{N} groups together form a 5- to 6-membered heterocyclic or heteroaryl ring;
each instance of X is, independently, —O—, —S—, or —N(R^{N})—;
each instance of Z is, independently, O, S, or N(R^{N});
each instance of R¹ is, independently, cyclic or acyclic, substituted or unsubstituted aliphatic; cyclic or acyclic, substituted or unsubstituted heteroaliphatic; substituted or unsubstituted aryl; or substituted or unsubstituted heteroaryl;
each instance of R² is, independently, hydrogen; cyclic or acyclic, substituted or unsubstituted aliphatic; cyclic or acyclic, substituted or unsubstituted heteroaliphatic; substituted or unsubstituted aryl; or substituted or unsubstituted heteroaryl; and
each instance of a is, independently, an integer between 1 to 6, inclusive;
providing a biologically active agent or a diagnostic agent;
mixing one or more compounds and one or more biologically active agents or diagnostic agents; and
tableting the mixture.

30. The method according to claim 29, wherein the compound of Formula I is in an amount greater than about 50 mg in the tableted material.

31. The method according to claim 29, wherein the tableting of step (iv) is by direct compression.
